(19) United States
(12) Patent Application Publication
(10) Pub. No.: US 2023/0340601 A1
(43) Pub. Date: Oct. 26, 2023

(54) Transcriptome Analysis For Treating Inflammation

(71) Applicant: Regeneron Pharmaceuticals, Inc., Tarrytown, NY (US)

(72) Inventors: Wei Keat Lim, Tarrytown, NY (US); Kaitlyn Gayvert, Tarrytown, NY (US); Sivan Harel, Tarrytown, NY (US); Jennifer Davidson Hamilton, Tarrytown, NY (US); Sara Hamon, Tarrytown, NY (US); Matthew F. Wipperman, Tarrytown, NY (US)

(21) Appl. No.: 18/306,737

(22) Filed: Apr. 25, 2023

Related U.S. Application Data
(60) Provisional application No. 63/334,837, filed on Apr. 26, 2022.

(51) Int. Cl. C12Q 1/6883; C12Q 1/6874

(52) U.S. Cl. CPC C12Q 1/6883; C12Q 1/6874; C12Q 2600/106

(57) ABSTRACT

The present disclosure provides methods of identifying a disease or condition suitable for treatment with dupilumab. The present disclosure also provides methods of identifying a subject having a disease or condition suitable for treatment with dupilumab. The present disclosure also provides methods of carrying out a clinical trial for dupilumab treatment of a disease or condition.

### FIELD

The present disclosure provides methods of identifying a disease or condition suitable for treatment with dupilumab, methods of identifying a subject having a disease or condition suitable for treatment with dupilumab, and methods of carrying out a clinical trial for dupilumab treatment of a disease or condition.

### BACKGROUND

Dupilumab, a fully human monoclonal antibody, blocks the shared receptor component for interleukin-4 (IL-4) and interleukin-13 (IL-13), which are drivers of type 2 inflammation in multiple diseases, including eosinophilic esophagitis (EoE). Dupilumab is indicated in the United States for use in subjects with uncontrolled moderate-to-severe atopic dermatitis, moderate-to-severe asthma with an eosinophilic phenotype or oral corticosteroid-dependent asthma, and inadequately-controlled chronic rhinosinusitis with nasal polyposis.

Pharmacogenetic tests, along with other information about subjects and their disease, disorder, or condition, can play an important role in drug therapy. However, such data typically relate to the clearance of small molecules where liver or kidney metabolism is well established, or certain definitive biologic markers are deemed as an indication of the disease, condition, or disorder. A caveat of such approach has been realized by the industry recently: typically, a disease, condition, or disorder, and the treatment thereof by a therapeutic agent, may involve multiple biologic molecules' small fluctuation, sometimes in an undefined biological pathway. Individually, each of such biologics may not exert a significant effect to the subject, but the accumulative effects of these biologic molecule level's fluctuation trend found in an individual patient, before or after the therapeutic treatment, may reveal the fitness of a particular therapeutic's treatment. Therefore, there is a long-felt but unmet need for methods to identify subjects that would benefit from treatment with any particular biologic therapeutic agent.

In some embodiments, the dupilumab treatment core gene signature is generated by determining differential gene expression of a dupilumab treatment group and a placebo treatment group for a plurality of treatment studies and identifying a plurality of genes that are differentially expressed.

In some embodiments, the clinical trial comprises generating a normalized enrichment score (NES) for the dupilumab treatment core gene signature prior to initiation of treatment of a subject with dupilumab and at least one time point after initiation of treatment of a subject with dupilumab.

In some embodiments, when dupilumab treatment results in a decrease in the NES for the dupilumab treatment core gene signature to an acceptable value, the clinical endpoint has been achieved.

### SUMMARY

The present disclosure provides methods of identifying a disease or condition suitable for treatment with dupilumab, the methods comprising: a) generating a dupilumab treatment core gene signature; b) screening the dupilumab core gene signature against a whole transcriptome profile from a plurality of disease studies; and c) identifying a disease or condition in the plurality of disease studies having a differential gene expression that is in the opposite direction from the dupilumab treatment core gene signature; thereby identifying a disease or condition suitable for treatment with dupilumab. In some embodiments, generating the dupilumab treatment core gene signature comprises determining differential gene expression of a dupilumab treatment group and a placebo treatment group for a plurality of treatment studies, and identifying a plurality of genes that are differentially expressed. In some embodiments, the plurality of treatment studies comprises eosinophilic esophagitis, atopic dermatitis, asthma, grass allergy, and chronic rhinosinusitis with nasal polyposis. In some embodiments, the genes in the core gene signature identified from the differential gene expression are selected as having a fold-change ≥2, and/or a q<0.05 in ≥3 out of 5 treatment studies. In some embodiments, the fold-change comprises subtracting the changes in expression in the placebo treatment group from the dupilumab treatment group. In some embodiments, the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab. In some embodiments, the differential gene expression for the asthma and grass allergy treatment studies are carried out by comparing the gene expression with allergen challenge to the gene expression without allergen challenge. In some embodiments, the differential gene expression is analyzed by a microarray or RNASeq. In some embodiments, the differential gene expression of the eosinophilic esophagitis, asthma, and grass allergy treatment studies is analyzed by RNASeq. In some embodiments, the differential gene expression of the atopic dermatitis and chronic rhinosinusitis with nasal polyposis treatment studies is analyzed by microarray. In some embodiments, the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes. In some embodiments, screening the dupilumab core gene signature against a whole transcriptome profile from a plurality of disease studies comprises: i) performing a differential gene expression analysis on the whole transcriptome profile for each disease study in the plurality of disease studies; and ii) generating a normalized enrichment score (NES) for all diseases in the plurality of disease studies using the plurality of genes that are differentially expressed and are in the dupilumab treatment core gene signature. In some embodiments, performing a differential gene expression analysis on the whole transcriptome profile for each disease study in the plurality of disease studies is performed for disease versus healthy controls. In some embodiments, the plurality of disease studies comprises the Gene Expression Omnibus database or the ArrayStudio DiseaseLand database. In some embodiments, the NES is generated using a gene set enrichment analysis tool that takes both positive and negative gene sets into consideration. In some embodiments, the NES is generated by: a) ordering the plurality of genes that are differentially expressed from the most positive (i.e., most up-regulated) to the most negative (i.e., most down-regulated) values to generate a ranked gene list (R+); b) identifying hits (i.e., the rank for genes in the core signature) independently for the positive (i.e., most up-regulated) gene set (S+) in R+, and the negative (i.e., most down-regulated) gene set (S−) in R−, wherein R− is the inversed ranking of R+ with inverted values; c) combining R+ and R− and reordering the values by keeping the hits for both S+ and d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r; e) determining an Enrichment Score (ES) as a maximum deviation from zero along the running score; f) repeating steps a)-e) with a random gene set for 1,000 times to compute the ES null distribution; and g) generating the NES as ES divided by the mean of ES null distribution. In some embodiments, the method further comprises computing the statistical significance by comparing the observed ES to the null distribution or sample label (disease/healthy) permutations. In some embodiments, step a) comprises using log 2 fold-change or z score. In some embodiments, R+ and R− are ranked by log 2 fold-change comparing the mean gene expression in disease samples to the mean gene expression in healthy samples. In some embodiments, the method comprises computing the NES for all disease studies using a ranked list for each disease study. In some embodiments, a diseases with significant NES is a disease suitable for treatment with dupilumab.

The present disclosure also provides methods of identifying a subject having a disease or condition suitable for treatment with dupilumab, the methods comprising: a) generating a dupilumab treatment core gene signature; b) screening the dupilumab core gene signature against a whole transcriptome profile from the subject; and c) determining whether the subject is suitable for dupilumab treatment. In some embodiments, generating the dupilumab treatment core gene signature comprises determining differential gene expression of a dupilumab treatment group and a placebo treatment group for a plurality of treatment studies, and identifying a plurality of genes that are differentially expressed. In some embodiments, the plurality of treatment studies comprises eosinophilic esophagitis, atopic dermatitis, asthma, grass allergy, and chronic rhinosinusitis with nasal polyposis. In some embodiments, the genes in the core gene signature identified from the differential gene expression are selected as having a fold-change ≥2, and/or a q<0.05 in ≥3 out of 5 treatment studies. In some embodiments, the fold-change comprises subtracting the changes in expression in the placebo treatment group from the dupilumab treatment group. In some embodiments, the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab. In some embodiments, the differential gene expression for the asthma and grass allergy treatment studies are carried out by comparing the gene expression with allergen challenge to the gene expression without allergen challenge. In some embodiments, the differential gene expression is analyzed by a microarray or RNASeq. In some embodiments, the differential gene expression of the eosinophilic esophagitis, asthma, and grass allergy treatment studies is analyzed by RNASeq. In some embodiments, the differential gene expression of the atopic dermatitis and chronic rhinosinusitis with nasal polyposis treatment studies is analyzed by microarray. In some embodiments, the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes. In some embodiments, screening the dupilumab core gene signature against a whole transcriptome profile from the subject comprises: i) transforming the whole transcriptome profile from the subject into z-scores; ii) ranking the z-scores; and iii) generating a normalized enrichment score (NES) for all ranked z-scores using the plurality of genes that are differentially expressed and are in the dupilumab treatment core gene signature, thereby representing the dupilumab signature enrichment for the subject. In some embodiments, the NES is generated using a gene set enrichment analysis tool that takes both positive and negative gene sets into consideration. In some embodiments, the NES is generated by: a) transforming each gene expression within the plurality of genes into a z-score, and ordering the plurality of genes that are differentially expressed from the most positive (i.e., most up-regulated) to the most negative (i.e., most down-regulated) values to generate a value of R+; b) identifying hits independently for the positive (i.e., most up-regulated) gene set (S+) in R+, and the negative (i.e., most down-regulated) gene set (S−) in R−, wherein R− is the inversed ranking of R+ with inverted values; c) combining R+ and R− and reordering the values by keeping the hits for both S+ and S−; d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r; e) determining an Enrichment Score (ES) as a maximum deviation from zero along the running score; f) repeat steps a)-e) with a random gene set for 1,000 times to compute the ES null distribution; and g) generating the NES as ES divided by the mean of ES null distribution. In some embodiments, the method further comprises computing the statistical significance by determining the 95^{th} percentile NES from healthy control samples. In some embodiments, the method comprises computing the NES for all disease studies using a ranked list for each disease study. In some embodiments, when the NES of the subject is higher than the NES of a healthy control, the subject is suitable for dupilumab treatment.

The present disclosure also provides methods of carrying out a clinical trial for dupilumab treatment of a disease or condition, the methods comprising using a dupilumab core gene signature as a clinical endpoint for the clinical trial. In some embodiments, the dupilumab treatment core gene signature is generated by determining differential gene expression of a dupilumab treatment group and a placebo treatment group for a plurality of treatment studies, and identifying a plurality of genes that are differentially expressed. In some embodiments, the plurality of treatment studies comprises eosinophilic esophagitis, atopic dermatitis, asthma, grass allergy, and chronic rhinosinusitis with nasal polyposis. In some embodiments, the genes in the core gene signature identified from the differential gene expression are selected as having a fold-change ≥2, and/or a q<0.05 in ≥3 out of 5 treatment studies. In some embodiments, the fold-change comprises subtracting the changes in expression in the placebo treatment group from the dupilumab treatment group. In some embodiments, the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab. In some embodiments, the differential gene expression for the asthma and grass allergy treatment studies are carried out by comparing the gene expression with allergen challenge to the gene expression without allergen challenge. In some embodiments, the differential gene expression is analyzed by a microarray or RNASeq. In some embodiments, the differential gene expression of the eosinophilic esophagitis, asthma, and grass allergy treatment studies is analyzed by RNASeq. In some embodiments, the differential gene expression of the atopic dermatitis and chronic rhinosinusitis with nasal polyposis treatment studies is analyzed by microarray. In some embodiments, the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes. In some embodiments, the clinical trial comprises generating a normalized enrichment score (NES) for the dupilumab treatment core gene signature prior to initiation of treatment of a subject with dupilumab and at at least one time point after initiation of treatment of a subject with dupilumab. In some embodiments, when dupilumab treatment results in a decrease in the NES for the dupilumab treatment core gene signature to an acceptable value, the clinical endpoint has been achieved. In some embodiments, the NES is generated by: a) ordering the plurality of genes that are differentially expressed from the most positive (i.e., most up-regulated) to the most negative (i.e., most down-regulated) values to generate a value of R+; b) identifying hits independently for the positive (i.e., most up-regulated) gene set (S+) in R+, and the negative (i.e., most down-regulated) gene set (S−) in R−, wherein R− is the inversed ranking of R+ with inverted values; c) combining R+ and R− and reordering the values by keeping the hits for both S+ and S−; d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r; e) determining an Enrichment Score (ES) as a maximum deviation from zero along the running score; f) repeat steps a)-e) with a random gene set for 1,000 times to compute the ES null distribution; and g) generating the NES as ES divided by the mean of ES null distribution. In some embodiments, the method further comprises computing the statistical significance by comparing the observed ES to the null distribution or sample label (disease/healthy) permutations. In some embodiments, step a) comprises using log 2 fold-change to compare gene expression after dupilumab treatment to gene expression prior to initiation of treatment with dupilumab. In some embodiments, a plurality of samples is obtained from the subject and the NES is generated for each sample.

The present disclosure provides methods of treating a subject having a disease or condition suitable for treatment with dupilumab, the methods comprising: a) identifying the subject as having a disease or condition suitable for treatment with dupilumab comprising: i) generating a dupilumab treatment core gene signature; ii) screening the dupilumab core gene signature against a whole transcriptome profile from the subject; and iii) determining whether the subject is suitable for dupilumab treatment; and b) administering dupilumab to the subject having a disease or condition suitable for treatment with dupilumab. In some embodiments, the dupilumab treatment core gene signature comprises determining differential gene expression of a dupilumab treatment group and a placebo treatment group for a plurality of treatment studies, and identifying a plurality of genes that are differentially expressed. In some embodiments, the plurality of treatment studies comprises eosinophilic esophagitis, atopic dermatitis, asthma, grass allergy, and chronic rhinosinusitis with nasal polyposis. In some embodiments, the genes in the core gene signature identified from the differential gene expression are selected as having a fold-change ≥2, and/or a q<0.05 in ≥3 out of 5 treatment studies. In some embodiments, the fold-change comprises subtracting the changes in expression in the placebo treatment group from the dupilumab treatment group. In some embodiments, the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab. In some embodiments, the differential gene expression for the asthma and grass allergy treatment studies are carried out by comparing the gene expression with allergen challenge to the gene expression without allergen challenge. In some embodiments, the differential gene expression is analyzed by a microarray or RNASeq. In some embodiments, the differential gene expression of the eosinophilic esophagitis, asthma, and grass allergy treatment studies is analyzed by RNASeq. In some embodiments, the differential gene expression of the atopic dermatitis and chronic rhinosinusitis with nasal polyposis treatment studies is analyzed by microarray. In some embodiments, the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes. In some embodiments, screening the dupilumab core gene signature against a whole transcriptome profile from the subject comprises: i) transforming the whole transcriptome profile from the subject into z-scores; ii) ranking the z-scores; and iii) generating a normalized enrichment score (NES) for all ranked z-scores using the plurality of genes that are differentially expressed and are in the dupilumab treatment core gene signature, thereby representing the dupilumab signature enrichment for the subject. In some embodiments, the NES is generated using a gene set enrichment analysis tool that takes both positive and negative gene sets into consideration. In some embodiments, the NES is generated by: a) transforming each gene expression within the plurality of genes into a z-score, and ordering the plurality of genes that are differentially expressed from the most positive (i.e., most up-regulated) to the most negative (i.e., most down-regulated) values to generate a value of R+; b) identifying hits independently for the positive (i.e., most up-regulated) gene set (S+) in R+, and the negative (i.e., most down-regulated) gene set (S−) in R−, wherein R− is the inversed ranking of R+ with inverted values; c) combining R+ and R− and reordering the values by keeping the hits for both S+ and S—; d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r; e) determining an Enrichment Score (ES) as a maximum deviation from zero along the running score; f) repeat steps a)-e) with a random gene set for 1,000 times to compute the ES null distribution; and g) generating the NES as ES divided by the mean of ES null distribution. In some embodiments, the method further comprises computing the statistical significance by determining the 95^{th} percentile NES from healthy control samples. In some embodiments, the method comprises computing the NES for all disease studies using a ranked list for each disease study. In some embodiments, when the NES of the subject is higher than the NES of a healthy control, the subject is suitable for dupilumab treatment.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows the top 30 genes with the greatest change in expression after administration of dupilumab 300 mg qw versus baseline. Transcriptomes for healthy and EoE patients reproduced with permission from Sherrill et al. (Genes Immun., 2014, 15, 361-69). All genes met significance thresholds. EoE, eosinophilic esophagitis; qw, weekly. Pink highlighting indicates positive upregulation and blue highlighting indicates negative downregulation.

FIG. 2 (Panels A and B) show an effect of dupilumab 300 mg qw versus placebo on the published EoE transcriptome in esophageal tissue at week 12. Panel A shows a correlation of the published EoE transcriptome and transcriptome post-dupilumab treatment. Panel B shows an effect of dupilumab 300 mg qw vs placebo on the expression of genes involved in pathways most significantly dysregulated in EoE: gene ontology analysis.

FIG. 3 shows type 2 inflammatory gene expression signatures in patients with EoE, atopic dermatitis, nasal polyps and asthma.

FIG. 4 shows the log 2 fold-change of the gene expression after treatment in each indication. Pink highlighting indicates positive upregulation and blue highlighting indicates negative downregulation.

FIG. 5 shows that ulcerative colitis (UC) NES is significantly enriched but not among the top 10. When NES was computed at an individual patient level using a UC study (GSE87466) with 87 patients and 21 healthy control, dupilumab NES were statistically significant in 33% of the patients.

FIG. 6 shows that in a CRSwNP study, a set of 25 genes (identified from treatment and clinical response signature) was shown to be more predictive of response to each CRSwNP clinical endpoint (CONG, NPS, CT-LMK, and UPSIT) than other available circulating biomarkers. A receiver operator characteristic analysis was used to assess the ability of the NES score representing the transcriptional signature and more standard biomarkers to discriminate the responders for each of the four major endpoints, as well as response across multiple endpoints. The predictive performance for each biomarker was summarized by calculating the area under the receiver operating characteristic curve (AUC). The dupilumab core gene signature highly overlapped with the 25 gene set and could be predictive of response to wider dupilumab indications.

FIG. 7 (Panels A and B) show long-term changes in gene expression profile in the dupilumab 300 mg qw group from TREET. Panel A shows change in EDP-NES from baseline to week 24 and week 52. Each lane represents a distinct patient. Panel D shows EDP-NES changes from baseline to week 24 were significant in both adolescents and adults. EDP, EoE diagnostic panel; EoE, eosinophilic esophagitis; NES, normalized enrichment score; qw, weekly.

FIG. 8 (Panels A-E) show effect of dupilumab on markers of cell proliferation, mast cell activation, T cells, and antigen-presenting cells in patients treated with dupilumab 300 mg qw or placebo. Panel A shows expression of cell proliferation marker MIB-1 (Ki67) (left) and change in the proportion of cells expressing MIB-1 (right) from baseline to week 12. Panel B shows expression of the mast cell serine protease tryptase (left) and change in proportion of cells expressing tryptase (right) from baseline to at week 12. Panel C shows expression of cytotoxic T cell marker CD8 (left) and change in proportion of cells expressing CD8 (right) from baseline to at week 12. Panel D shows expression of helper T cell marker CD4 (left) and change in proportion of cells expressing CD4 (right) from baseline to at week 12. Panel E shows expression of antigen-presenting Langerhans cells marker CD1a (left) and change in proportion of cells expressing CD1a (right) from baseline to at week 12. The change in the percentage of positive cells from baseline for the placebo and dupilumab immunochemistry images was quantitated using HALO image analysis software. Nominal P values were calculated using unpaired two-sided t-tests to compare the absolute change from baseline between the placebo and treatment groups. NS, nonsignificant; qw, weekly.

FIG. 9 shows Dpx3 response signature in EoE ph3.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician or a veterinarian.

As used herein, a list comprising A, B, “and/or” C provides: (i) A alone; (ii) B alone; (iii) C alone; (iv) A and B; (v) A and C; (vi) B and C; and (viii) A, B, and C. Thus, a list comprising A, B, C, . . . , and/or N has n constituents, where n is a positive integer provides all possible combinations of A, B, C, . . . N up to and including a combination of all n constituents.

As used herein, the phrase “opposite direction” in the disease signature, refers to a comparison between disease samples and healthy samples. A gene is up-regulated (red/pink) if the expression is higher in the disease, compared to healthy. In the treatment signature, a comparison is between post-treatment and pre-treatment. A gene is down-regulated (blue) if the expression is lower after treatment, compared to baseline (before treatment). “Opposite direction” refers to genes that significantly changed in opposite direction in disease and treatment signature, e.g. up-regulated in disease (compared to healthy) and down-regulated after dupilumab treatment (compared to before treatment).

In the process of drug discovery, it is beneficial to identify existing approved drugs effective to new indications (be it a disease, condition, or disorder), or a subset of patient population that would better respond to the existing drug. In traditional drug effect studies, single gene analysis upon drug treatment (i.e., t-test) for each individual gene is utilized to identify suitable biomarkers to a target, and to alter such biomarker's level to achieve a presumed treatment effect. This approach, however, may lead to the creation of a long list of statistically significant genes without connecting their biological relevance. In addition, such gene lists among different clinical studies may have little overlap; and most importantly, they may miss the pathway effects of each gene involved in the disease, condition, or treatment effects. For example, several small gene expression changes, although individually not significant in t-test, may have more collective impacts on a given disease or condition, than one gene that changes quite a bit but has negligible effects on the disease or condition. To avoid such an outcome, a genome wide approach is developed in this disclosure, wherein a core gene signature set in a drug's clinical study is first identified, compared with a whole transcriptome profile of interest, to obtain a normalized enrichment score for use to identify an appropriate indication or patient population to respond to the drug, particularly dupilumab.

The present disclosure provides methods of identifying a disease or condition suitable for treatment with dupilumab. In some embodiments, the embodiments comprise generating a dupilumab treatment core gene signature. In some embodiments, the methods comprise screening the dupilumab core gene signature against a whole transcriptome profile from a plurality of disease studies. In some embodiments, the methods comprise identifying a disease or condition in the plurality of disease studies having a differential gene expression that is in the opposite direction from the dupilumab treatment core gene signature. In some embodiments, the methods identify a disease or condition suitable for treatment with dupilumab.

In some embodiments, the methods comprise generating the dupilumab treatment core gene signature comprising determining differential gene expression of a dupilumab treatment group and a placebo treatment group for a plurality of treatment studies. In some embodiments, the methods result in the identification of a plurality of genes that are differentially expressed.

In some embodiments, the plurality of treatment studies comprises eosinophilic esophagitis, atopic dermatitis, asthma, grass allergy, and/or chronic rhinosinusitis with nasal polyposis. In some embodiment, the plurality of treatment studies comprises eosinophilic esophagitis. In some embodiments, the plurality of treatment studies comprises atopic dermatitis. In some embodiments, the plurality of treatment studies comprises asthma. In some embodiments, the plurality of treatment studies comprises grass allergy. In some embodiments, the plurality of treatment studies comprises chronic rhinosinusitis with nasal polyposis. In some embodiments, the plurality of treatment studies comprises any disease, disorder, or condition, involving or suspected to involve IL-4 and/or IL-13.

In some embodiments, the genes in the core gene signature identified from the differential gene expression are selected as having a fold-change ≥2, and a q<0.05 in ≥60% of treatment studies. Alternately, the use fold-change threshold of 1.5 or a p value (instead of q value) can be used. In some embodiments, the genes in the core gene signature identified from the differential gene expression are selected as having a fold-change ≥2 in ≥60% of treatment studies. In some embodiments, the genes in the core gene signature identified from the differential gene expression are selected as having a q<0.05 in ≥60% of treatment studies

In some embodiments, the fold-change comprises subtracting the changes in expression in the placebo treatment group from the dupilumab treatment group. For example, fold-change is the average expression in group 2/average expression in group 1.

In some embodiments, the differential gene expression for eosinophilic esophagitis, atopic dermatitis, asthma, grass allergy, and/or chronic rhinosinusitis with nasal polyposis treatment studies are carried out by comparing the baseline gene expression before treatment with dupilumab to the gene expression after treatment with dupilumab. In some embodiments, the differential gene expression for eosinophilic esophagitis is carried out by comparing the baseline gene expression before treatment with dupilumab to the gene expression after treatment with dupilumab. In some embodiments, the differential gene expression for atopic dermatitis studies is carried out by comparing the baseline gene expression before treatment with dupilumab to the gene expression after treatment with dupilumab. In some embodiments, the differential gene expression for asthma treatment studies is carried out by comparing the baseline gene expression before treatment with dupilumab to the gene expression after treatment with dupilumab. In some embodiments, the differential gene expression for grass allergy treatment studies is carried out by comparing the baseline gene expression before treatment with dupilumab to the gene expression after treatment with dupilumab. In some embodiments, the differential gene expression for chronic rhinosinusitis with nasal polyposis treatment studies is carried out by comparing the baseline gene expression before treatment with dupilumab to the gene expression after treatment with dupilumab.

In some embodiments, the differential gene expression for the asthma allergy treatment studies are carried out by comparing the gene expression after allergen challenge to the gene expression before allergen challenge. In some embodiments, the differential gene expression for the grass allergy treatment studies are carried out by comparing the gene expression after allergen challenge to the gene expression before allergen challenge.

In some embodiments, the differential gene expression is analyzed by a microarray or RNASeq. In some embodiments, the differential gene expression is analyzed by a microarray. In some embodiments, the differential gene expression is analyzed by a RNASeq. In some embodiments, reverse transcription polymerase chain reaction (RT-PCR) can be used to measure gene expression.

In some embodiments, the differential gene expression of the eosinophilic esophagitis, atopic dermatitis, asthma, grass allergy, and/or chronic rhinosinusitis with nasal polyposis treatment studies is analyzed by RNASeq. In some embodiments, the differential gene expression of the eosinophilic esophagitis treatment studies is analyzed by RNASeq. In some embodiments, the differential gene expression of the atopic dermatitis treatment studies is analyzed by RNASeq. In some embodiments, the differential gene expression of the asthma treatment studies is analyzed by RNASeq. In some embodiments, the differential gene expression of the grass allergy treatment studies is analyzed by RNASeq. In some embodiments, the differential gene expression of the chronic rhinosinusitis with nasal polyposis treatment studies is analyzed by RNASeq.

In some embodiments, the differential gene expression of the eosinophilic esophagitis, atopic dermatitis, asthma, grass allergy, and/or chronic rhinosinusitis with nasal polyposis is analyzed by microarray. In some embodiments, the differential gene expression of the eosinophilic esophagitis is analyzed by microarray. In some embodiments, the differential gene expression of the atopic dermatitis is analyzed by microarray. In some embodiments, wherein the differential gene expression of the asthma is analyzed by microarray. In some embodiments, the differential gene expression of the grass allergy is analyzed by microarray. In some embodiments, the differential gene expression of the chronic rhinosinusitis with nasal polyposis is analyzed by microarray.

In some embodiments, a gene of the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and/or RNF103-CHMP3, or any subset thereof comprising at least 40 genes, at least 42 genes, at least 44 genes, at least 46 genes, or at least 47 genes.

In some embodiments, a gene of the plurality of genes that are differentially expressed comprises LRRC31, SLC45A4, BUB1, PARP12, TMEM154, AL831977, INPP4B, C2orf72, ALOX15, RASGRP1, WFDC5, CLSTN3, HBP1, LUZP6, SMCR7, KCNJ5, SLC26A4-AS1, VGLL1, PARVB, BCL6, PHACTR1, IFRD1, OR13A1, MAB21L3, TNFAIP6, IFIT2, AX747171, DNM3, DVL1, FBXO27, KIAA2022, RNF224, CLC, GATA3, AK093551, EXOG, KIAA0226, FAM86DP, CA12, AL157440, CCL26, TENM3, TSPAN15, DTX3L, REEP6, EME2, HMOX1, C9orf169, POSTN, KLK1, DERL3, APBB1IP, ATP1B1, TMPRSS11E, RPS6KL1, MAMDC2, SLC26A4, PDLIM3, INFRSF10D, ATP8A1, ZNF382, HIST1H2AC, KCTD17, EEF1A2, NTRK1, CLDN5, PROCA1, CPT1B, C1orf122, CNKSR3, GATSL3, S100A12, PMCH, KIF21B, GALNT6, GBP2, C7orf73, MYOM3, CAMK2N1, MYEOV, PTCHD4, SLAMF1, TLR2, CADPS2, MOSPD1, UCKL1-AS1, AX748345, SRGAP1, NRXN1, CXCR4, IL3RA, ITGA4, CRAT, FAM46B, HBB, YOD1, NEFL, GGH, OAS2, AX748379, EFNA2, TUBA4A, LRFN2, RHBG, CXCL1, AK124308, LINC00672, AK092098, HMOX2, LZTS1, PHGDH, SNX9, ATP13A5, ARHGAP25, BTN2A3P, NR5A2, ARHGAP28, SPRR1A, AGFG2, RNF180, NEFM, TSPAN3, GNRH1, PSMB10, ARHGEF26-AS1, SBSPON, RAB17, ISM1, TNIP3, NFE2L3, SEMA3A, PRC1, CNNM1, CARHSP1, PLIN3, VSIG2, CTSG, C5orf56, ELOVL5, FBXO6, TOLLIP-AS1, C4orf3, CDKL5, ZNF101, DKFZp686O16217, CD36, PPAP2C, COPZ2, AK026714, AK128153, WNT9A, LPIN1, IGH2, ADAMTS14, VAMP5, LOC115110, PEBP1, DNM1, TMEM61, CAP2, abParts, STAB1, DLGAP5, OSCAR, RNF11, STAR, UCA1, KRT4, IL1RL1, LAIR1, SH2D2A, NCKAP1L, AK289390, NUDT8, MYH11, LY6K, CPA3, CHST2, MPEG1, STARD4, AKR1C3, HEG1, PPP1R36, ALDH3B1, CXCL6, PSMB9, TIGIT, TFRC, JUN, PRSS27, KRT13, TP53TG3D, TREML2, MAPK10, GVINP1, HNRNPU-AS1, MORN4, CECR7, TSPAN7, MTUS2, LURAP1L, HLF, MYOF, NAIP, BC063600, FAM126A, LINC00482, HIST1H2BC, HDC, LIFR, TMPRSS4, ATP12A, PCDHGA1, ZNF662, STX1B, XCR1, AK128525, ADRA2A, ARMCX3, FAIM3, TPGS1, PPFIA4, SOSTDC1, HP07349, Ig kappa, BDNF, FAM69A, KLRAP1, ROMO1, MPZL3, ME1, TCN1, TMEM71, RUFY4, CCDC146, FAM13A-AS1, BSPRY, AK127124, MT1E, LOC646329, EMR1, APOL4, CLU, C11orf92, AHNAK2, MRC2, PLEKHA6, PER1, CDH26, MDK, IGF2BP2, LIPA, SNX16, CEACAM3, PKNOX2, FOXI2, IGJ, CEP290, BC144457, CLNS1A, RNF10, KIFC3, PTPN20A, BNIP3, LOXL4, POU2AF1, DFNA5, GTSE1, GBP6, ENTPD2, TRIB3, STXBP5-AS1, SUSD2, C11orf93, AK095112, TPX2, MGST1, PLLP, SLC16A9, SYT17, GPR97, CTSK, MAP3K14, IQCK, ALDH3A1, LINC00319, GMDS, ABCG2, IL19, LINC00939, FAM111B, IFNAR2, KRT31, ANKRD36BP1, BARX2, CWH43, MS4A2, LITAF, INFRSF10A, ERG, HSFX2, DMPK, TREH, ACADL, SIGLEC6, ADAM8, ERCC6L, GLB1L, ZNF582, EM L5, AIFM2, PCDH20, KCNJ16, P2RY14, HLA-B, MCAM, SERHL2, SERHL, GPSM1, XYLT1, SLC9A3, FETUB, HCK, SERPING1, SH3GL1, CCL17, FAM83A-AS1, AKR1B15, TRPM6, FLJ00157, CDC25C, AK091624, CDKN1A, WNK4, ENC1, DQ571917, EMILIN2, FBXL2, ATP2A3, DL490846, JUND, DZIP1, BC036236, ZYG11A, C1QTNF1, BX647608, HCP5, DKFZp547K2416, EGOT, OAZ3, SH3BP5, FAM171A2, APOBEC3A, USP32P2, CSGALNACT1, BAK1, SGTA, ZNF416, TMEM38A, EDNRB, COL6A6, LOC619207, UBASH3A, CXCL16, SUCO, IL22RA1, CAPN5, ZNF92, CNTN4, LTA4H, AF268386, PRICKLE4, RFFL, RNF169, QSOX1, PLK3, BCL2L15, HS3ST1, APOBR, FE5, DOPEY2, PMM1, C18orf25, KCP, BMPR1B, LOC100506714, CDC45, BC013821, ARG2, C15orf48, SLC15A2, MAP1LC3A, ANO1, PARP14, DEPDC1, CTSS, NCKAP5, UBTD1, PLEKHF1, TFAP2B, IL8, SH3RF2, SEMA6B, ARSB, NOV, NBPF16, FAM129B, HIST4H4, CDK15, PDZK1IP1, BTN3A2, AKAP2, RBM10, ACOT11, KLK5, DUSP13, DPP4, ICAM1, CHN2, ODF3B, NCK2, CD68, TRIM56, PLCXD2, TPSAB1, HEPHL1, ASB2, N4BP2L1, SALL2, AKAP6, AK093892, ZBED2, CXCR2P1, ABCC5, HDX, EVI2A, MLXIP, SYT8, SPECC1, HIST1H2AD, DKFZp667J0810, IQGAP2, GNLY, PDPR, TSPAN13, AK127120, UACA, BC020196, IL9R, SERPINE1, TSHZ3, RASA3, RAP2B, C8orf47, CPLX3, SULT1B1, C20orf197, NCF1C, MAP3K8, EBLN2, FAM149A, TMEM86A, RANBP9, ZDHHC15, RPTN, CHST7, SYCP2, ERI1, FRMD6-AS1, PPP1R15A, ANKRD20A11P, ENPP4, UBD, IL36G, ZC3H12A, TBC1D1, FAM222A, GSTA4, HLA-DQB2, FAM43A, GLDC, ANTXR2, TOP2A, PARP3, SCNN1D, TENM4, KIF26A, SLC6A15, MMP12, SDPR, MYO3A, INFRSF10B, DNAJB5, AK4, TTC40, SESN2, WBSCR17, CCDC150, GCH1, RAB23, MUC1, KIAA0232, PTK6, KRT7, IGLL5, CCR1, IL18BP, ZC3H12D, SLC2A4, CTTNBP2, TOM1, CHST6, ADAMTS15, CTAGE15, DMD, SRGAP3, DCLK1, FBXO34, TMEM57, KRT6B, TPSB2, CTLA4, C1QC, FAM46C, ZNF117, AHDC1, AX747793, KBTBD12, CD200R1, OLFML2B, SEMA3B, TLR8, ANKH, S100A14, LRRK2, CYP26B1, PRRX1, CYP4X1, CCL2, LINC00673, KYNU, RHCG, C2orf16, SLITRK5, ATP4A, GLCCI1, OASL, GLIPR2, CNPPD1, RNF222, GJA1, AX746725, SAMSN1, PSTPIP2, MYL9, ZNF702P, PFKL, LINC00152, GPR1, MYO1H, CD209, GEMIN8P4, MMP14, SRPX, BNIPL, FBXL22, BC013229, ACOX2, SIGLEC17P, PABPC4L, CENPF, MAP1B, TMEM79, HBA1, CC2D2A, CST6, PHLDB2, ICAM5, GPR39, AX747833, CLDN8, CCL20, ZNRF1, SPRR3, APOBEC3A_B, PARP8, BIK, TIFAB, BCKDHA, SNX24, CALML3, PPP2R2C, COL6A5, PTPN7, FAM101B, CCDC39, TMEM127, VPS37B, CCNG2, KRT6C, OLAH, PDE10A, KRT23, AJ515158, YPEL3, ISG20, PARK2, PORCN, KCNJ2, COL3A1, ALOX5AP, RGS20, BICD1, FRAS1, HES4, ALPK3, GATA2, PLA2G3, SAM D4A, IFNGR1, TNIP1, FLJ11235, SH3TC2, TSPAN12, P2RX1, PRIMAL, PTGS2, RIC8B, LOC646862, PLA2G4F, DEPTOR, BC048201, NTRK2, LCP2, AK093534, C4orf21, CYP27A1, CCDC110, DKK4, SMTNL2, IGHG1, LOC100507463, CLEC2B, LRPSL, SYNE4, BTBD19, B3GNT8, CLVS1, CTSC, ESR1, BIRC3, SP140L, SPNS3, AK056396, MIF, FAM107B, AK023178, C1orf74, IL411, GLRX, C1orf170, NSG1, ARHGAP44, HIST1H4E, MGAT3, AK098438, SPTBN1, ACAP1, MDFIC, PAPPA, ACPP, BC033949, APOL1, IL15RA, SEC62, CYTIP, CRYL1, SPINK5, GPLD1, CXCL2, CCL24, CD84, FA5, CD28, SH2D3A, ARHGAP10, UBL3, ISM2, CCDC68, CD44, LAPTM5, PPP1R12B, ABCA3, TNNI2, USH1G, TMEM37, SECTM1, CCDC24, MB21D1, NLRC3, SAMD4B, ARID3A, PDZD4, DDAH1, RTP4, STK17B, RASSF6, LCK, C1orf210, RUNDC3B, GATSL2, COX7A1, LHFPL2, TAP1, EFCAB4A, CLDN23, LIPE-AS1, FAM35DP, TRAM1L1, DEGS2, AKAP12, GPR183, PIM2, GJA5, PPP2R2B, NAMPT, VARS2, IL18, LTF, PPM1H, DENND3, USP12, BAIAP2, TAX1BP1, SLC16A6, AADACL2, MSLN, P2RY10, AK054970, ZC3HAV1L, SEC14L2, SDCBP2, AX747393, SH3GL3, ITGA2B, NMI, COL6A1, SOAT1, CBX4, SPHK1, ESYT3, AK056618, AJ606330, IF130, LRRC6, EVA1C, RABGGTA, S100A6, ETV1, LYPD2, RUNX2, CRMP1, AK128252, NEK2, ANXA11, ASCC2, AHCY, RGS11, SPON1, XAF1, FAM24B-CUZD1, RDH10, ETV5, ULK3, ANKRD36C, ERO1L, KCNH2, C1RL-AS1, RNF183, PARVG, CDC34, SCEL, PABPC1, RASGEF1B, SERPINB4, HCST, IL27RA, DAB2, LOC100130705, TMEM217, RMND5A, CRNN, PRKD1, IF144, SERPINB10, AK125301, LRG1, FAM214A, MGC39372, RASAL2-AS1, HRH1, SYT2, NDC80, VPS13C, CCSER1, CLEC4F, PPL, TDRD5, SYNPO, DAPK2, BTG1, PTBLP, FAM161B, SULT2B1, ELN, PAQR8, LOX, RHOH, LRP12, ITGA1, MN1, CCDC157, CXorf57, AK095621, HPGDS, TRANK1, GABRP, CAMK1D, CPPED1, IER3, SBSN, BTNL9, IGF1, ADAMTS9, AK097957, AURKB, UNC13D, KRT1, SMCO4, DSC1, SAA1, LILRB1, NEK3, PRR11, NHSL1, KIF21A, GYLTL1B, LINC00443, TSPAN1, THBS1, GBP4, ITGAE, RPS6KA6, CDKN2B, ACADM, RDH5, LBH, STRIP2, PRSS12, NUSAP1, UPRT, VSIG10, TMC1, RFK, EPPK1, SKAP1, CD33, LOC100506100, C20orf24, ARHGEF26, LOC646999, SLCO4A1, PDCD1LG2, P2RY1, CELSR3, BTG3, STXBP1, SLC13A4, ZNF726, UNC93A, CFI, EGLN3, NEDD9, LINC00865, FAM134B, CYP4F3, BOC, TP53INP2, LYPD6B, KRT16P2, DL491896, TMC8, GNA15, ASPG, SFTPD, CNFN, CHL1, MMRN1, C10orf10, DKFZp686M11215, STK33, SNCG, WNK1, C21orf15, CMYA5, CCDC34, C1QTNF3, PITRM1-AS1, FAM222B, NAPRT1, DGAT2, AGAP11, COL4A4, LOC100129550, ALDH1L2, TRGC2, SH3D19, PYGB, PHLDA1, SAMD5, SOCS3, HLA-F, PLCE1, LPCAT4, MKNK2, CASP10, LOC100653515, MAPT, SLC28A3, AK123771, MMP28, SMC2, ITPRIP, GPRC5D, STEAP4, SPNS2, TESPA1, STAT4, MELK, PLK1, HEBP2, C10orf99, GRIN2A, MMEL1, RGS1, RBMS3, NCAPH, CLEC4A, MEGF9, LCA5, PDGFRA, RBM11, PAMR1, FN1, USP54, CCDC74A, DNAJC5, SEC24C, NUCB2, VASN, DOC2B, RNF207, ANLN, DOCK11, WNT4, NTF3, PI4K2A, IL36A, LAX1, GBP1, CLUAP1, PIEZO2, N4BP3, PRSS8, SH3BGRL2, ANKRD20A9P, F13A1, GIMAP2, SFXN3, DLC1, SEC14L1, NCCRP1, NOX5, DUSP5, GRAP2, IFITM1, CENPI, TNFAIP8L1, FAM86EP, CHAC2, MT1X, ESAM, KCNJ2-AS1, ASB9, ROBO1, CKS2, SPINT1, MAPK8IP1, HOGA1, FW340055, ADAMTS1, FAM124B, ARHGAP15, MMD, EGLN2, DSC2, FSBP, BEAN1, LOC645638, DUSP10, ASS1, DHX58, SLC6A9, IMPA2, HCAR1, GRPEL2, CA2, CASP7, L3MBTL1, FADS1, FBXO32, PTN, KRT79, ACHE, CREB3L1, HLA-C, IL16, TMEM229B, IGLON5, SRPX2, CYP2W1, LCE3E, SGK1, TNRC6C-AS1, LAG3, SH2D1A, DHRS2, LOC100505702, BEX4, DES, CFB, IFFO2, IF16, ITGAL, TMEM65, PLEKHG6, NRG4, AL122050, ADRBK2, ATF3, TOP1P1, TLDC2, PPP1CB, PLEKHM1, FAM86B1, HIST1H2BF, P2RY6, B2M, AL831889, NPEPPS, DHRS4L2, EFNB3, STRADB, BC015433, AX747758, TG, MET, THSD4, TMED3, WNK2, AX747150, NRTN, TRIM17, TRAF3IP3, OLR1, NREP, FKBP8, BC02, ROR1, GPR78, PP7080, DDX60L, ARHGEF37, RRAGD, HIST1H3F, ARHGEF4, SLC6A8, CCBE1, SLC2A3, ITK, KIF4A, IL1RL2, FAM83A, RDH12, CX3CR1, LOC399715, CD300LF, LOC100506123, CASC5, C7orf60, PLA2G4B, DEAF1, SPSB3, ECM1, HAS3, FARP1, ADA, SARM1, HSPB8, HIST1H2BJ, C15orf59, UPK2, TIMP1, VDR, CD52, TOR1AIP2, ZNF556, MERTK, PLEKHG4B, DIRAS1, TPK1, AK126693, CEP19, KIAA1683, STXBP6, RAP2A, B3GNT7, SPRR2A, IL18R1, SH3PXD2B, HSD17B11, MGC2752, AX748015, DAGLA, SOWAHA, AX747544, KIT, SMCO2, HDAC9, ZSWIM4, BC133670, AF143871, TF, TREX2, MRC1, SLC7A7, IL32, PDE6A, NCOA1, SRGAP2C, A2ML1, LOC100289511, LIF, CASS4, NBEA, CAPS, NAGK, WDR26, SPAG1, CPZ, EMR4P, TRAF5, CAPN3, ANKRD20A4, ARSG, ZNF185, TRIP10, ZNF426, CYP2S1, KIAA1456, KBTBD11, SV2A, UBE2E2, HOPX, SDR9C7, PADI1, IGFBP3, BC040358, DQ573949, BC019880, PDZD8, BC040935, MAP1S, PANX2, SIDT1, AP4B1-AS1, AK127443, NPAS1, LRMP, SLC2A4RG, UGT1A7, KRT78, CACNB4, IL2RG, APOD, HRNR, XYLB, SLC27A6, FAM20A, GADD45B, C2, TMEM98, SLC16A2, PLAGL1, TYRO3, PDLIM2, ZNF578, HIST1H3D, IF135, KLRB1, A4GALT, CMAS, MCOLN3, TUBB6, OTOP2, AGPAT9ANKDD1B, N4BP2L2-IT2, CEP152, BCOR, AKR1C1, PLCXD1, FAM174B, RNF182, CBRC7TM_40, AK092331, KIF20A, TPRG1L, PCLO, METRNL, LOC96610, ANKRD37, PTGES, CD9, HUNK, TEC, ZNF800, SEPT3, VSIG10L, MT2A, CD163L1, SLFN13, LAT, PLB1, PCBP1, PNMA1, NQO1, KRT40, HNF4G, CLMN, NUP62CL, SOCS6, TMEM40, ARHGAPS-AS1, ZNF667-AS1, MYRF, CH25H, PRRSL, TAGLN, BC039551, RPL23AP82, WASF3, IVL, AK097184, SLC6A14, GAB3, ANKRD36B, OXR1, FAM78Bl, CLECSA, FUT11, TP53I11, PRG2, SLC27A2, KCND1, LGMN, ATP6VOC, PLCD3, MXD1, CDA, PGBD5, KLHL5, SFMBT2, LNX2, MXRA8, FAM122C, BC039389, SYT7, CDH3, GPR153, EIF3CL, SCRIB, SCAND1, FFAR4, BC131755, NRG2, NEK6, CTAGE4, AF157115, SORBS2, TMEM254, CCL21, C9orf66, AX747517, SRGN, FCER1G, LOXL2, CHI3L1, CYP2C18, B4GALT5, AMFR, ADIRF, BC040701, MC1R, C9orf40, PCNXL2, SLPI, PIH1D2, AMACR, RNF103-CHMP3, GFI1, PLAT, TYROBP, PKDREJ, RNF24, LINC00887, PDZRN3, NPC1L1, EMR2, COL1A2, STIL, ZSCAN18, SCNN1G, TP53BP2, RAP1GAP, AK123263, BX649158, FA2H, ZNFX1, KCNK5, DLGAP1-AS1, RTN4RL1, PDE1A, FTH1, ITGAM, ZNF503-AS2, PPARGC1B, DIS3L2, FBXW4P1, ATG9B, HIST1H2AE, AFF3, SDK1, DUOX1, IQGAP3, S100A10, UNC13B, PAPL, ILIA, CPA4, LINC00707, POC1B, IF116, CX3CL1, NCR3LG1, LINC00933, TRIM2, RHOF, ITGA9, TNFRSF18, MTFR2, IGFBP2, IL36B, FLJ23867, FAM109A, FER1L6, VCAM1, C1orf216, IFITM3, KCTD11, SMIM5, ADM2, CXCR2, EML1, SLC44A4, SULF1, FAM3C, BC038574, SDK2, BAI1, SPIRE1, TAS1R3, SLC45A3, BTN3A3, AX746775, AREG, S100A16, CXXC5, CHRNB4, LRRC20, RASGRP4, ARNTL, GZMA, SPAG4, BOK, ZNF460, CDH22, GSTT2, TFPI, AL832891, AK308965, SERTAD1, PWWP2B, WWTR1, TPTEP1, BC017578, LRRC8D, NFATC2, AX747179, CDR2L, SERTAD2, AKR1B10, LOC729970, SPTBN4, SCUBE2, SERPINA1, BC034424, MVD, VMAC, AL110181, PELI1, HTR3B, NCF1B, FAM26F, SHCBP1, PCDHGA5, SYNGR3, WNT2B, CEACAM7, SPRR2D, NCF1, FLG2, KPNA2, MAP1LC3B2, FBXO39, CITED2, SASH1, LINC00664, KITLG, ZNF487P, UK, HIST1H2BK, ABHD12, ACTG1, MYZAP, CYP2B7P1, GALNT4, BTN3A1, BC069212, ANKRD6, KLB, RNF128, PKIB, CPNE4, IL17RB, F2R, ZNF827, ARMCX5-GPRASP2, USP6NL, HIST2H2BF, CECR6, HS3ST6, MICB, FBN1, PDE4D, INPP5A, AK130329, RND3, AK096475, CRYAB, CD69, RAC2, GMFG, CAPS2, TSPYL4, VAV1, MGLL, UPK1A, GPRCSB, C3orf52, CD37, SH3BP5L, AK094188, RP1L1, FAM180A, PPP1R3G, ARHGEF6, BST2, CLDN1, TBKBP1, EDIL3, C15orf62, S100A8, GNA14, CFH, COL6A2, MAN1A1, HMGCS1, ZFAND5, ZNF254, VIPR1, CYP4F35P, NPR1, GIMAP1-GIMAP5, TMEM133, SP6, TM4SF19, EFNA5, AK097370, SMOX, CNRIP1, TNS4, DKFZp686B07190, BC040700, ITGBL1, DBNDD1, TMEM8A, SLC38A4, PKP2, MSRB1, SKA1, LINC00265, FER, CYP2C9, TGM1, CGNL1, TNFSF13, PRODH, BUB1B, TSPO, ZNF425, ZNF430, TNNT1, ETNK2, CSF2RB, RGS18, RGS16, SRXN1, ABHD5, PCSK5, KLRG2, HMGCS2, ADAM28, HCG26, PIK3AP1, ZDHHC2, GCNT1, ADAM11, EPHA10, SPRR2B, GPR160, ORAI1, PLEKHG2, PRSS22, TAB3, CTIF, EPS8L1, HSPA2, KLHL6, CD274, FMO4, TEF, PPM1K, SHROOM4, BCAR3, ZNF812, TMEM176B, PARP9, AK123332, SYAP1, HIST2H2BE, ST3GAL4, ZNF667, TGM3, CECR2, IL18RAP, PCCA, XXYLT1, GLTP, ANKS1B, ATP6V0A4, KRT77, NYAP1, RGS2, LOC283070, NAPA, CSRNP3, NELL2, CD207, NHLH2, CMKLR1, TGM2, LIMA1, SAMD10, ARL4C, CPT1C, GCHFR, PAK7, PLAUR, MTHFD1L, KIF23, MARCKS, GLTPD1, SPRY1, MT1L, OR7E91P, HTR2B, EPSTI1, ASPM, PANK1, FAM83D, KCNAB1, TTC22, PRRT4, SCIN, RAD51AP2, IRF1, TEX9, ZNF703, GATM, USP13, ENDOU, PMP22, ISLR, FAM83E, NINJ2, PRKACB, COL21A1, RHOD, DKK1, IFIT3, BID, C1QA, LRIG1, NUP214, SNPH, MAGED4, HCN2, ABCA12, ECT2, WIPF1, PHACTR4, KIAA0355, ST6GALNAC1, LYNX1, CALB2, BTK, AMICA1, TMED7-TICAM2, LFNG, CNN3, FAM3D, SLC46A2, SLURP1, RSAD2, EPHA4, BC063788, CLCF1, FAM102A, TUFT1, ICOSLG, RUSC2, EDAR, UBE2L6, BC051760, ITM2A, BC040684, FCER1A, FIBIN, DLGAP1, CEP72, ABCA8, AK095366, ALKBH7, ATP6V1C2, B3GNT3, IL1RN, CTNNAL1, COL8A2, CYP2R1, CLEC2D, CCDC85B, GUCY2D, IL36RN, SLC9A3R2, AR, ADORA3, GPR143, SLC18A2, PANK2, CEACAM19, MFSD7, S100A9, IGFL1, GCNT3, CD300A, KRT17, ALDH5A1, CCDC127, TMEM80, SLC16A3, NDUFA4L2, SFRP1, CYTH4, KIF2C, SIRT7, KIAA1244, TOB1, HIST1H1C, EPHA6, DPYD, GPR82, TYMS, C15orf52, C17orf103, FAM86FP, LOC100127983, CR936677, LOC100129034, ICOS, LAT2, TNS1, C1orf21, TMOD3, ARHGAP27, EPB41L3, MMP25, CTSW, UBA7, BDKRB1, LTBP2, CD1A, CYP4F22, NKPD1, C1orf110, RAB20, CCNB1, METTL21A, CHMP4C, LOC401052, HERC2P4, KRT32, RAB44, TLR4, SCIMP, FOXO3, ITGB5, ADCY9, UPK3B, AQP7P1, FCGR3A, GSDMB, GPR68, TEAD1, SNX8, DHRS1, NAP1L2, NGEF, MMP9, CBWD5, KIF14, MYL12A, DMTN, FSCN2, ZNF555, LGI3, OR2A7, ADAMTS2, DDX26B, PIP5K1C, AK056780, COBL, MAL, C3orf80, LILRB2, SLCO3A1, FOLH1, SDC1, TPRN, IL34, HHIPL1, ANKRD20A5P, AADAC, FAM46A, AK123872, GALNT14, SLC10A6, BAIAP3, SNX21, RNASE7, PPARGC1A, NCF4, SELL, GCAT, CAMSAP3, KRT33A, CDHR1, LOC100127888, POC1B-GALNT4, HJURP, TCIRG1, GPD1, ARHGAP39, PADI3, DAPL1, AIF1L, CMPK2, PSMB8, UBE2C, MACROD1, BCAS1, CAB39L, LNX1, C1orf168, SPP1, CHST15, CDK1, JUNB, CLEC3B, AK125699, SLC6A11, RNF223, SLC15A1, LIMD2, KIF18A, COL7A1, FKBP1A-SDCBP2, SHROOM3, DUSP4, FLG, MEOX1, IKBKAP, FJX1, FOSL2, AVPI1, CLIC3, DHRS9, BAIAP2L2, RNASE1, ORAI2, CSF1, NMRK1, RTN1, CDKN1C, POU3F1, ST6GAL2, PXDN, ATP8B4, CLIC6, DOCK5, RAB36, PIK3C2G, SLC35F3, ALOX12, ANKRD65, MX1, GCSAM, SEMA4D, DUSP14, LCN2, FADS6, NKX6-2, RYR3, CD226, AK123584, MAP2K1, FAM84A, PITX1, C7orf57, C1orf177, MFHAS1, WDR52, PTPRB, CYP2U1, ARRDC1, TNFRSF11A, TMPRSS11B, FAM25C, SLC16A1, NFKBIE, RELB, CPNE5, SLC2A11, HOMER2, UNC79, HTR3A, ZSWIM5, CADM1, MMP19, ITPRIPL2, EREG, DYNAP, TSC22D2, SLC13A5, CAPN14, NTN1, GALNT5, RCOR2, LPIN2, FBXL16, HNMT, RAB6B, STOM, TMTC3, CCNB2, KRT18, KPNA7, LDHD, BC033124, CHAC1, CYP7B1, IFIH1, FANCI, MYRIP, CSRNP1, LOC100132891, TUBB2A, RGS17, SHF, SLC39A8, RALGAPA2, SRCIN1, AMDHD2, SLCO2B1, VIT, DSG1, GAPT, PLIN5, LINC00900, PQLC1, NDRG4, CACNA1G, RNF39, BC107108, TRIM22, LOC100288637, CHST11, DEF8, AK311374, TTC9, TMEM74, ABTB2, OSMR, CEL, PLCB2, AK125212, ZFPM1, AX747104, NPPC, PSCA, AKAP5, CAPN13, NTF4, FAM167A, VSIG8, LOC100996255, PCBP1-AS1, SLC6A1, SELP, FAM118B, APOBEC3D, FHOD3, ELL2, ABHD16B, TMIE, NR1D1, NCF2, SLC9B2, CD53, LOC285074, FABP5, VWA2, TMEM105, RHCE, TMEM253, DENND4A, FAM111A, DQ586822, ZNF519, SERPINB1, KDM5B-AS1, AK097161, TMEM176A, FLT3LG, SLC38A5, KIF1C, PINK1, MAPK3, SLC7A5, MC5R, DDX60, LY75, LYZ, XK, NPR3, SCNN1B, GRB14, DKFZp667P0924, C12orf75, RNF213, CD48, HOTAIRM1, PAX9, SRGAP2D, SEPT5-GP1BB, CRCT1, C3AR1, LOC729603, SLC22A3, ABLIM2, H2AFJ, NOXO1, KRT3, MUC21, VWA5A, GPR65, NKG7, FCRLB, C10orf12, DOCK3, GP6, SAMD11, IL15, IL7R, IGFBP7, HSPA6, FZD7, CYP2C19, TICAM1, CLDN10, LONRF2, AK055623, PITRM1, ACSL6, NETO2, PKP3, BC046483, ZNF365, NRP2, ATP10A, CDCA2, NFKBIA, ZNF208, SPTBN5, SPRR2E, IRX6, TMEM173, JAK3, SMAD1, CD164L2, HIF1A, AX747832, GGTA1P, LOC284551, NAV1, THY1, SPAG5, AURKC, RIOK3, DTX2, C6orf132, ACER1, CD22, ITGAX, ATP11C, LOC100129195, GDPD3, PMAIP1, TCP11L2, CES1P2, C12orf54, PDE4A, PLAU, RASGEF1A, COL14A1, LINC00675, C19orf26, LGR6, FOXE1, GIPR, SLC38A6, AK126286, LOC344887, LOC100288181, AKR1C2, BC070322, CD101, FGF1, MEIS1-AS3, MAPK7, ALDH1L1, PRKAA2, CDK14, ANKRD62, PDE3B, FASLG, ARHGAP9, DHCR24, TST, KCNMA1, BPGM, SPINK7, COL6A3, ABCA10, CTSL1, CAST, COL18A1, AK311497, GPX3, KPRP, UPK1B, C1S, DOCK2, JARID2, DBN1, TGFB1, ZNF714, BC042588, DDX58, FPR1, IKZF1, LOC730101, WWC1, DMRT2, HIF1A-AS2, LRRN2, GPR34, DSE, APCDD1, DQ576756, ID1, PIM1, LINC00346, AK054845, CISH, NFIL3, LINC00173, NDUFA6-AS1, TMEM63C, CSNK1E, CSTB, IGSF11, GCNT2, LY96, FAR2, AMER1, SLC34A3, MROH6, SYT15, C4orf6, LINC00839, EDNRA, PTP4A3, FUT3, CTNNBIP1, UBALD1, EMP1, ANKRD31, RARRES3, AURKA, PCOLCE, SSBP3, CSK, MBLAC1, SCN2B, CRISP2, CHST9, VSIG4, LINC00511, SIPA1L2, PALMD, TIMP2, ADM, MYOZ1, IF144L, HMMR, DKFZp686D16130, RAB40C, MMP24-AS1, RECQL4, THRA1, CRYM, GRK5, SASH3, RRM2, KIAA0913, SH3RF3, ERV3-1, C12orf56, REN, CBLN3, DOCK7, PBK, DIRAS3, PXDC1, RASAL1, LDLRAD1, EPGN, IF127, C1QB, CCNA2, NFKBIZ, PRDM1, GFOD2, CGREF1, EDN2, DNAH11, IRF9, SCARA3, LGALS3, SERPINB9, CCDC69, MAFF, HSN2, MFAP3L, LOC646214, FPR3, NDUFB11, ADIPOR2, PACRG, SERINC2, PSORS1C2, APOL2, KLK7, LAMP3, PLA2G4A, HTR7, BC044939, RNF208, PEG10, PIK3R6, DOK3, SERTAD4, RCAN3, AK8, CABLES1, HIST1H4H, HCG22, TCERG1L, PIF1, APOL6, ARRDC3, CCL22, ZFP36, SORBS1, EDN3, COL12A1, MDGA1, ZNF37BP, PRDM4, CCDC6, C4orf19, RBP7, GYS2, SIGLEC1, AEBP1, TUBBP5, SAMD9, ABLIM1, DUOXA2, HIST1H2BG, GGT8P, ID3, DNAH17, BC150585, LLGL1, BLVRA, BC068095, NBPF10, ARG1, ZEB2, ZMYND15, ABHD4, HECA, ABHD6, BC041455, PFKFB3, DKK2, RARB, SLC15A3, SAV1, ZDHHC20, PLEKHN1, FCGBP, ZNF431, FAM25A, SHC2, KCNMB3, RASL11A, ABO, HK2, THSD7B, PHACTR2, FNDC4, CD163, PCP4L1, CENPW, EHD1, ELF3, FAM228B, LINC00628, BC024173, NR4A1, MIAT, BLM, PFKFB2, SLIT2, SFT2D2, CRIP2, PRLR, NUP210, HAPLN3, PRELID2, NME4, PTK2B, PFN2, ABLIM3, MUC22, RAB37, FOXQ1, TRIM21, AK095151, ITPKC, GHR, LRRC24, SFTA2, APOBEC3B, CAMK1, AOAH, EPN1, TMEM52, CXCL14, AK097590, DPCR1, CHI3L2, SCCPDH, ACSL4, GJA3, LINC00920, SLC5A10, MIPEPP3, RTP1, APOL3, CD40, PDPN, SYTL5, PHC1, BBOX1, EEPD1, CIDEA, MUC4, ETV7, TMEM91, ADAMTS17, SLC37A2, MALL, NKX2-8, CES1, ASPN, BIN2, MFSD4, PRDX5, TRPS1, LRRC37A4P, LINC00568, HILPDA, SIGLEC10, TFEC, STON1, FAM221A, GTPBP2, AIM1L, DISP2, GCKR, C21orf88, FMO1, SMPDL3B, MAF, GALE, UFSP1, AL832737, KRT2, TMC5, COL1A1, SERPINB3, HDAC5, RGCC, BC064974, C3orf67, LOC286297, AQP7, AK096066, SOX2-OT, AX748273, UBAP1L, EPS8L2, CLDN17, MT1H, PRKXP1, AX747264, AB209185, EPCAM, RPS6KB2, FUT6, UPP1, MT1G, AIM2, RANBP17, SGOL2, LRRC16B, DBP, SH3RF1, TP5313, PNLIPRP3, ST6GAL1, PLSCR4, NUF2, UNCSB, HIPK2, GPR111, H19, SLC8A1-AS1, LTBP1, FUT8, XPR1, MAP3K9, NDOR1, CNST, PPDPF, SYNPO2L, CCBL1, IGFBP6, CCDC82, CCT6B, ADSSL1, MACROD2, OSBP2, SPINK8, ANPEP, CDH11, EPAS1, PARD6B, CFTR, ANKRD20A3, SERPINA11, CRISP3, LUM, OLFML3, SEC14L1P1, KLC3, GYPC, BLVRB, CCNYL1, CRTAC1, SERPINE2, BC041347, SIRPG, PDGFD, MARCH1, MRVI1-AS1, TNFAIP8L3, LAMB4, PRTFDC1, CEP55, EPHA3, OPTN, SLC4A4, LOC100130476, ACOT4, KRTAP3-2, ACSL5, CENPE, LOC100130557, SPRR1B, NR1D2, AK056431, KCNQ5, FSTL1, MK167, LOC100506472, KATNBL1, CNTNAP2, THEM5, MARCO, PRICKLE2, ACOT9, SLC9A2, ZNF770, MCF2L2, CSTA, and/or IL12A. In some embodiments, any one, any two, any three, any four, any five, any six, any seven, any eight, any nine, any ten, any eleven, any twelve, any thirteen, any fourteen, any fifteen, any sixteen, any seventeen, any eighteen, any nineteen, or any twenty of the genes of the plurality of genes that are differentially expressed set forth above can be omitted from the gene expression analysis.

In some embodiments, the plurality of genes that are differentially expressed does not comprise MEOX1, ARHGEF6, GPR34, PIK3R6, STK17B, CAMK1, CNRIP1, IFIT3, AK055623, WFDC5, ADAM28, FBXL2, NFIL3, FOXQ1, CEP55, VAMP5, CCL2, MELK, SHCBP1, RRM2, PSMB10, BAK1L, KIT, SLC45A3, ZSWIM5, KRT16P2, GPR143, DDX58, RARB, LIMD2, PLIN5, C1QB, SLC16A2, CXCL16, CBRC7TM_40, PSMB9, SLC7A7, CD9, ECT2, SLC15A3, ARHGEF37, DNM3, AK098438, HLA-F, HLA-B, CDC45, FAS, SCIMP, IL18R1, MICB, NFE2L3, BDNF, SDPR, TNFRSF18, DSE, DLGAP5, KPNA2, BC069212, SKA1, MEIS1-AS3, C3AR1, APOL3, RUFY4, RGS18, TYROBP, TCIRG1, GALNT5, FPR3, ZC3HAV1L, PLK1, AX747758, CTLA4, TAP1, GPR183, CYP2R1, ORAI2, DENND4A, SCCPDH, CENPE, CDC25C, FJX1, SAV1, CHST15, SLC39A8, FLT3LG, HMMR, CENPW, CD101, SH3PXD2B, KRT23, ADA, UBE2C, FAM46A, ETV7, FAM111B, MMP14, NDC80, AADAC, CCR1, RAC2, HCK, GABRP, KIF4A, ABHD4, PRELID2, COL6A6, BC040701, NAV1, FCER1G, POC1B, PRODH, LY96, ACOT9, BUB1, PPAP2C, FAM69A, ERCC6L, MTFR2, C1QA, PRR11, GFI1, MFAP3L, SHC2, ACSL5, PSMB8, LRP12, IFITM3, IRF1, GTSE1, AQP7, LOC100506714, CASP7, C1QC, CASC5, CCNB1, GPR68, CDCA2, LAMP3, SGOL2, NUF2, SMC2, TCERG1L, MUC4, PTPN7, NCAPH, CEP19, TAGLN, C9orf40, CCNB2, SPP1, SELP, DDX60, HCST, LOC100129550, ICOS, RANBP17, SEMA6B, KIF2C, CCNA2, COPZ2, TBC1D1, TRIM22, C5orf56, CDK1, PBK, RDH10, PARP8, SMCO2, GPR82, DEPDC1, TOP2A, MB21D1, HUNK, TTK, RYR3, FSTL1, CXCR4, GLCCI1, CLMN, IFIH1, DOK3, OASL, BC038574, GPR39, ZNF827, NEK2, ST6GAL1, GATA3, CTAGE4, DUOX1, HJURP, TMPRSS4, SEC62, EFCAB4A, ANLN, KIF20A, KIF18A, LOC100506100, CLEC4A, VWASA, MAPK10, AMICA1, CD300A, PIF1, PARVB, LAT2, SPAG5, LTBP1, IL15RA, RHOH, IKBKAP, CHN2, BTG1, FAM83E, LINC00900, PRICKLE4, TLR8, AURKB, ABCA12, STRIP2, B2M, TMEM98, SULF1, INFRSF10A, CENPF, CD52, PITRM1, XPR1, LIPA, CLNS1A, TPX2, ARSB, FES, IFIT2, PPM1H, ASB2, PIM2, STIL, RGS16, PIK3AP1, LOC115110, LPCAT4, COL6A5, HNF4G, ZEB2, CD274, MTHFD1L, TFEC, MK167, KIF23, ASPM, FBXO6, IL15, HLA-C, SASH3, FM01, CENPI, ERI1, OLFML2B, LY75, INFRSF10D, DMD, SPTBN1, BUB1B, CHST11, LYZ, AOAH, PRC1, FAM46C, PARP14, CEL, RASL11A, OSCAR, PDE10A, LCP2, PLAT, CYTH4, APOBR, PLEKHG2, LIMA1, CLIC6, CFH, TNRC6C-AS1, TG, SFXN3, A4GALT, ODF3B, PRRX1, OR2A7, APOL4, BST2, MYL9, TYMS, STARD4, CD69, CHST9, CASS4, FLG2, NCF4, IGFBP6, HCP5, ATP2A3, LOC283070, KIF14, BLM, NUSAP1, C11orf93, C1RL-AS1, PRR5L, RAB20, ITGAX, GALNT6, MYOF, GCH1, SEMA3B, PPARGC1B, SLC38A5, PARP12, SOAT1, KIF21B, CD84, AK123771, RNF207, PROCA1, CLUAP1, IF116, CSF1, MFSD4, UBE2L6, MX1, ATP8B4, IQGAP3, PCCA, ATP11C, BCL6, ZC3H12D, ABCC5, SLFN13, HDX, RASSF6, ROBO1, GMFG, SMAD1, PLAU, PDPN, PDE3B, ANTXR2, CCDC150, LOC100507463, C1orf216, FAM101B, NUP62CL, UBA7, FANCI, CTSL1, IQCK, AKAP2, ITGAE, ZNFX1, CLDN1, USP12, C3orf52, EPSTI1, CD40, PLSCR4, SH2D2A, LAPTM5, NBEA, APOBEC3D, PXDN, CTSW, HDAC9, CTSS, PARP3, EVA1C, CCDC74A, IQGAP2, GBP1, EDNRA, IL3RA, ARMCX3, BC013821, IFNGR1, MC1R, ARNTL, IL7R, TLR2, BTN3A2, GNLY, LAT, ACSL4, ADAMTS9, FUT8, MPEG1, AK097957, ANKRD36B, SERTAD4, SCARA3, CAMK1D, PARP9, GPR65, DOCK7, BIN2, IF16, INFRSF10B, SERPINE1, LOC100288637, GIPR, IF144, BTN3A3, ZNF487P, TLR4, IRF9, ASS1, TMED7-TICAM2, NTF4, RAB23, BX647608, TRANK1, USP54, HSD17B11, PDE4D, PTP4A3, MYO3A, CD37, SLC38A6, BTG3, SLAMF1, RNF213, CEP152, RALGAPA2, PLCB2, CD53, APCDD1, CLSTN3, SKAP1, THY1, PDE4A, KCNMB3, FAM3C, WIPF1, LINC00173, TRIM21, TFRC, MCAM, EPHA4, CAPN13, CLU, TMEM91, TRAF3IP3, HCG26, DTX3L, NCKAP1L, GIMAP2, MDGA1, FAR2, APBB1IP, ERG, EVI2A, CD28, XAF1, DENND3, ARHGAP15, FOLH1, GLB1L, RIC8B, LINC00865, TIGIT, IGF2BP2, IL32, CADPS2, KCND1, APOL6, TMC8, CTSK, ESR1, BTN3A1, ATP10A, ARHGAP9, SIRPG, ATP8A1, RASA3, DAB2, IL18BP, MAN1A1, ATP12A, GLRX, DDX60L, ALOX5AP, LRRC6, PRSS12, LINC00673, TRAF5, COL6A1, LAG3, DOCK2, IKZF1, EPAS1, IFITM1, OAS2, SAMD4A, IFNAR2, DOCK11, COL12A1, CD226, NEDD9, L3MBTL1, SFMBT2, STON1, SRPX, C4orf21, PARVG, BC144457, TSHZ3, SMPDL3B, KIAA1456, GAB3, GSDMB, LOC729603, JAK3, DFNA5, NEK3, EXOG, KLRB1, CCDC146, CCDC82, CPT1B, N4BP2L1, SRGAP3, CYTIP, CD33, KBTBD11, SIGLEC1, AK123584, SLC22A3, CELSR3, IL16, FAM111A, ZNF702P, ACAP1, IL18RAP, GZMA, GCSAM, LRPSL, PRKXP1, MAP3K8, LOXL2, CD48, ITGA4, PDPR, P2RY10, PCP4L1, LINC00672, C10orf10, PCOLCE, SERPINB3, ITK, TMEM133, DLC1, GBP2, FAIM3, NAIP, GLIPR2, ASB9, COL6A2, AEBP1, OLFML3, BTN2A3P, SYCP2, AK054970, COL6A3, NKG7, STAT4, EIF3CL, DDX26B, ZNF37BP, COL1A1, GBP4, DL491896, LINC00939, FN1, RAD51AP2, CBWD5, WDR52, FMO4, SP140L, NLRC3, BC063788, LINC00511, PPP1R12B, AX747171, AK128252, PLCE1, PTPRB, KLRAP1, PTBLP, BC040358, MIAT, EBLN2, LCK, GNRH1, GVINP1, CLEC2B, ALDH1L2, CAPN3, NR5A2, VPS13C, C1S, AF157115, N4BP2L2-IT2, F2R, AF268386, FAM24B-CUZD1, FASLG, AX747179, SOX2-OT, LOC100506123, FARP1, AK093551, UBASH3A, ABCA10, AK127443, CLEC2D, AK095366, AK092098, TIFAB, IGFBP7, MAP1B, AL831889, RNF183, AK308965, AK092331, CECR2, FBN1, ISLR, CDH11, PTGS2, DKFZp686B07190, GIMAP1-GIMAP5, C11orf92, FAM13A-AS1, BC051760, AX747833, AK095112, ITGA1, LUM, LOC646214, SLC9A2, LOC619207, DQ573949, LOC100506472, AX748379, TOP1P1, LOC100130557, AK091624, DL490846, FGF1, AK093534, APOD, COL1A2, AK123872, C1QTNF3, AX746775, COL3A1, AK125301, ASPN, HNRNPU-AS1, SERPING1, AK123332, TRGC2, IF144L, ADAMTS2, SEMA3A, SEC14L1P1, USP32P2, TUBBP5, PITRM1-AS1, DKFZp686M11215, RBMS3, EPHA3, PIEZO2, CSGALNACT1, AL832891, BX649158, ABCA8, FCRLB, TPGS1, SCNN1D, NFKBIA, MYH11, HES4, AK126286, SYNGR3, SPNS3, SCAND1, ZNF578, JUNB, NELL2, BC041455, PRSS22, BC068095, TGFB1, KLK5, TPTEP1, PCDHGA5, PQLC1, RASGEF1A, ZFPM1, OTOP2, LOC100129195, CECR7, CPT1C, DUOXA2, C1orf122, ROMO1, SGTA, ZNF460, EPN1, BC064974, ALKBH7, DUSP14, MVD, TEF, NDUFB11, COX7A1, TNIP1, FBXW4P1, HIST1H2BJ, CDR2L, CCDC85B, and/or CYP27A1, or any combination thereof.

In some embodiments, the plurality of genes that are differentially expressed does not comprise CDC34, ZNF703, BC019880, BC063600, VSIG8, OPTN, ADCY9, SERTAD1, MACROD1, SRCIN1, SPRR1B, BICD1, DNAJB5, AMDHD2, DEAF1, ZNF726, GJA3, SLC2A4, NME4, CCL22, EHD1, ETV5, SLC6A9, ATP6VOC, UBTD1, PITX1, JUND, MUC1, EGLN2, PPP1R15A, SNPH, TSPO, AURKC, NBPF16, METTL21A, CD164L2, PCBP1, TTC40, MARCO, SV2A, ITM2A, SYNE4, AX748015, BOK, KRT18, RAB40C, LGALS3, LLGL1, LOC646862, CCDC157, VAV1, KCTD17, DIS3L2, PIP5K1C, LOC100130705, STXBP6, ARHGAP44, XXYLT1, DVL1, LRG1, C17orf103, RAB36, NAPRT1, GCAT, ZNF556, NETO2, ZNF208, ARID3A, GATSL2, NBPF10, KIF1C, RPL23AP82, DBP, OAZ3, CDKN1A, GLTPD1, CNTNAP2, METRNL, MIF, SDC1, KLC3, HMOX2, FBXO32, NUDT8, MRC2, PFKFB2, RBM10, TMEM127, RABGGTA, MAPK8IP1, SRXN1, RAP2B, FRMD6-AS1, BAIAP2, FKBP8, RPS6KB2, FAM122C, ITPRIPL2, FAM222A, LZTS1, ENTPD2, PTPN20A, STEAP4, BPGM, SCRIB, LFNG, NFKBIZ, PCDHGA1, MLXIP, CBX4, PWWP2B, TPRG1L, SH3BP5L, SYAP1, NAPA, RCAN3, AK095151, NCK2, DLGAP1-AS1, ARRDC1, PTK2B, LOC100996255, BC040700, TEX9, ABLIM2, RNF10, SH3GL1, HSFX2, CSRNP1, ANKS1B, KCTD11, REEP6, C1orf210, GNA15, IER3, PLCD3, BLVRB, PCNXL2, GALNT14, GPD1, BCKDHA, PLA2G4B, FKBP1A-SDCBP2, SLC34A3, UBALD1, MAPK7, LRRC16B, TPRN, C10orf12, MMP24-AS1, ITPKC, NDOR1, FAM86DP, SLC2A4RG, HIST1H2AE, SAMD10, KIAA0913, EPCAM, MEGF9, FAM86EP, MXRA8, FBXL22, ACTG1, CX3CL1, AREG, ZDHHC2, MARCKS, MYL12A, SIRT7, MYRIP, ACSL6, JARID2, PFKL, SH2D3A, STXBP1, DHRS4L2, DZIP1, SPHK1, CHAC2, CABLES1, ANKRD36C, S100A10, CCT6B, ANXA11, SPINT1, SERTAD2, DMTN, CHMP4C, RTN1, EME2, KCNK5, SSBP3, MAF, SAMD4B, ARL4C, FAM83D, TUBA4A, YPEL3, UPRT, DNAJC5, DBN1, PXDC1, TMEM86A, BTBD19, SDCBP2, PLCXD1, CYP2W1, ILIA, DKFZp667P0924, SPAG4, PANK1, FOXO3, CYP2U1, HSPA6, HECA, UNC5B, PARD6B, PEBP1, EGOT, WWC1, NR1D2, C4orf19, HBB, DEF8, HDAC5, KIAA0226, CNNM1, BSPRY, UBE2E2, NDRG4, AK311374, MBLAC1, RPS6KL1, GPSM1, SLC9A3R2, TMEM74, CDK14, PRDM4, SERHL2, SEC14L1, HSPB8, H2AFJ, PINK1, UFSP1, PER1, ZSWIM4, ANKRD20A4, IGFBP2, LINC00265, CPNE5, DQ576756, MORN4, UNC13D, FAM222B, AVPI1, TMEM63C, PMAIP1, HIST1H4E, HMGCS1, ARMCX5-GPRASP2, LRIG1, ZDHHC20, MAP3K9, RNF11, ZNF800, VMAC, CXXC5, RND3, TMEM80, LCN2, ZFP36, SIPA1L2, PDGFD, CNPPD1, FAM134B, TMED3, FZD7, DNM1, TUBB6, NINJ2, CLCF1, MAP2K1, XK, HOTAIRM1, SALL2, C20orf24, ISG20, PNMA1, LNX2, DHCR24, PLA2G4A, PRDX5, CEACAM19, FBXO27, C15orf48, PLA2G4F, CSNK1E, SOCS6, TEAD1, PDZD8, HIST2H2BE, FAM102A, STAR, HIST2H2BF, B3GNT7, HP07349, LGMN, TMEM79, CRYL1, SEC14L2, RNF24, CCDC127, COL18A1, FL11235, RUNDC3B, EFNB3, B3GNT8, AHCY, OXR1, TBKBP1, PHACTR4, ATP1B1, TSPAN13, HEBP2, S100A16, ITGB5, SULT2B1, CTIF, ZSCAN18, SP6, PANK2, RCOR2, ARHGEF26-AS1, FAM161B, MKNK2, KIAA1244, ARHGAP39, TST, AK8, FAM46B, AHDC1, VPS37B, IMPA2, FOSL2, ZNF770, TMEM40, HIPK2, PRSS27, ZNF430, AX748345, KIF26A, PLEKHG4B, XYLB, AK094188, GLTP, GUCY2D, SNX8, ADIPOR2, SERPINB9, HBA1, PRSS8, PKP3, DTX2, ZNF382, JUN, TSPYL4, CAMSAP3, CSK, ABHD6, ELF3, TMEM52, GTPBP2, LINC00319, PAPL, BC046483, RFFL, WNT4, NUP214, MFSD7, CGREF1, C7orf73, FAM149A, NAGK, TYRO3, ID1, PLIN3, HIST1H2BK, SNX16, ZNF582, ZFAND5, ABHD5, LDHD, SFT2D2, LOC646999, FAM20A, NDUFA6-AS1, N4BP3, PPP1CB, CCDC6, GALE, ASCC2, FFAR4, WWTR1, NOXO1, INPP5A, FER, FAM84A, CNKSR3, SPRR1A, PIK3C2G, MROH6, SPECC1, LOC285074, FUT3, DIRAS3, ARHGAP28, GBP6, TMEM65, KIFC3, S100A14, DBNDD1, MAP1S, THRA1, BCOR, SH3D19, DMPK, AKAP6, PLEKHG6, C9orf66, IL1RL2, NHSL1, PLEKHN1, IFRD1, CCDC110, TP53BP2, TTC9, SRGAP2D, KCNMA1, RNF208, ENPP4, RRAGD, PLB1, BDKRB1, AMER1, AK289390, NCKAP5, ANKH, FAM83A, PCLO, ZNF425, HEG1, SYT8, PDLIM2, CDKN1C, MRVI1-AS1, FAM78B, PLLP, ASPG, CASP10, RDH5, CAPS2, CAST, MDFIC, USP6NL, SLC10A6, BLVRA, ADSSL1, SEC24C, RTN4RL1, GFOD2, CCDC69, SPSB3, BC131755, CMAS, ANKRD6, HBP1, C8orf47, NSG1, PLEKHM1, BAI1, RNF128, TMOD3, SLC6A8, TMEM8A, RHOD, PKDREJ, NMRK1, AK130329, TAB3, CTNNBIP1, TRPS1, WNK2, RDH12, UPP1, ABHD12, KIAA0355, UBAP1L, ARHGEF4, LINC00887, EFNA5, FAM86FP, SMCR7, KRT79, TEC, SAM D9, AKR1C3, MGST1, NOV, ITPRIP, LINC00152, S100A6, LOC401052, FAM129B, TRIM56, PDZD4, GYLTL1B, FAM109A, PKIB, HIST1H4H, FAM221A, PAX9, PYGB, AL110181, PLEKHF1, RNF224, ARRDC3, SLC2A11, PRDM1, GYPC, PMM1, ARHGAP10, CLIC3, RECQL4, BC039551, AX748273, TMEM154, NCOA1, CLEC3B, C15orf62, UCA1, C19orf26, SERINC2, PHACTR1, AHNAK2, ABCA3, RIOK3, MYOM3, CLEC5A, TIMP2, MT1X, LINC00568, FOXI2, DOCK9, SUCO, UCKL1-AS1, MPZL3, TAX1BP1, WNT2B, GATM, LINC00675, CXorf57, CECR6, C6orf132, ABO, MOSPD1, ARG2, ELL2, HTR7, RAP2A, SRGAP2C, TOB1, TMIE, TICAM1, FHOD3, KATNBL1, CPPED1, UNC13B, C1orf21, MARCH1, CITED2, TUFT1, AIFM2, KLRG2, TUBB2A, SLC7A5, COL7A1, HIST1H3F, CNN3, HIST1H2AC, CD68, AK4, SNX24, FBXL16, MACROD2, HK2, EPS8L2, DOPEY2, SMIM5, SLIT2, IL22RA1, AK127120, FAM214A, NCCRP1, ARHGAPS-AS1, DHRS1, HCAR1, HIST1H1C, POU3F1, ADM, LINC00628, ARSG, RNF169, ADM2, BAIAP3, AIM1L, HMOX1, C15orf52, TNS1, SLPI, NCR3LG1, KLB, AK127124, FBXO34, RPS6KA6, TMEM254, SNCG, RP1L1, CNST, C4orf3, WASF3, AX747104, RAB17, SMCO4, BC039389, BNIPL, B3GNT3, PPDPF, SNED1, FAM95C, CXCR7, CLCA3P, C7orf55-LUC7L2, LINC00551, ARHGAP20, MB, FAM13C, SLC1A3, OR7E14P, ADARB2, AK093443, SPTSSB, AK128563, ANGPT1, FMO2, RBP1, CCL14, NWD1, TRPC1, ZNF471, ZFP28, OCA2, GNE, CTH, SFRP2, FAM181B, AX747081, AGR2, PDE4C, CACNA2D3, NHS, UGT1A9, BC027846, GSTM3, OXGR1, LINC00202-1, MROH7, TUB, PLCD4, B4GALNT4, CD177, TEX101, ABI3BP, CDKL2, TNFSF9, PLEKHG1, AK128534, RTEL1, WNT5A, AK128619, NAALADL2, PGLYRP4, FLJ41484, GLDN, CLCA4, RIMBP2, COL4A6, PMFBP1, TNNT3, CD302, AK127224, AK094577, FGF14, AQP5, SPRR2C, B3GNT6, BC010924, GSTA1, CYP3A4, LHX4, C17orf61-PLSCR3, FMO9P, PLSCR3, IL23A, NOS3, IGFBP5, FCGR1A, APOE, SLC26A9, KRT14, RRAD, SEMA3C, APOC1, GABRA4, PLA2G4E, TPPP3, NFKB2, C8orf44-SGK3, AL049415, CCDC28B, and/or DKFZp58661922, or any combination thereof.

In some embodiments, a gene of the plurality of genes that are differentially expressed comprises ANKRD20A12P, SGSM1, TRIM39-RPP21, MT1F, LRRc37A, HIST2H2BA, HYDIN, CAMK2B, IFNE, TSNAX-DISC1, CXCR1, AK300387, DQ587119, LOC100499484-C9ORF174, PLA2G4D, AK316321, X69637, SLC38A3, KCNQ3, LRRC37, FLJ22184, ZNF625-ZNF20, NOG, GDF7, TMEM189-UBE2V1, LOC400891, ARC, LPA, TDRG1, CDH20, C18orf61, MT1M, LOC441178, LOC149086, LMAN1L, AQP7P3, MT1A, MUC5B, TGM6, CCR3, SHD, SIX2, C6orf223, LOC154092, CEBPE, LINC00092, C12orf28, NFE2, MZB1, CLDN24, ELK2AP, HK3, CLDN22, FAM110B, FW340046, GATA1, MGC45800, SLC5A8, CR1, MGC32805, IL5RA, SOCS1, FOXD1, RGS13, TREM2, LIPG, STAP1, TTLL13, LOC100506585, GFRA2, ENPP3, CAPN8, FAIM2, SAA2, HAS2, OSM, DEFB104B, KIAA0125, LILRA6, AK124806, C9orf84, EGR2, CD1B, SIGLEC8, SNORA21, SIRPB1, SIGLEC7, MKX, ADAM12, C1orf167, CD300LB, APOC4-APOC2, ICAM4, LILRB5, IL13, BATF3, DBC1, C1orf228, BC075797, HRH2, COL4A3, AFF2, EXOC3L4, GCSAML, CD180, TNNC1, GFRA3, BC045578, CCDC60, DDO, GALR2, C1orf186, PITX2, APOC2, SLC2A6, CCL11, CD79A, TMEM92, NPY4R, TNFRSF4, RPPH1, DPEP2, TPSD1, HSP90AB4P, CHST1, C2CD4C, MUC16, CDHR3, AK125558, ASRGL1, LYPD1, C4orf48, LILRB4, CRB2, SAMD14, C19orf38, DQ658414, LRRC25, CD300C, FOXD2-AS1, TMOD1, CYP1B1, RENBP, CCDC19, CYP4Z1, LILRA1, CCDC170, HSD17B2, HAGHL, P2RX5, PTGDR2, BCL2A1, USP30-AS1, LYL1, CCRL2, IL2RA, CD1D, ERN2, MISP, BCL2L14, AK126744, ST8SIA6, KLHDC7B, TESC, AX747756, URGCP-MRPS24, LBX2-AS1, AATK, CTRL, CLNK, and/or RET, or any combination thereof. In some embodiments, any one, any two, any three, any four, any five, any six, any seven, any eight, any nine, or any ten of the genes of the plurality of genes that are differentially expressed set forth above can be omitted from the gene expression analysis.

In some embodiments, the plurality of genes that are differentially expressed does not comprise HRNR, MAP1LC3B2, TOLLIP-AS1, DCLK1, C1orf170, CCSER1, STK33, IGLON5, DHRS2, BC133670, FBXO39, ITGBL1, LINC00920, MCF2L2, AL831977, SBSPON, ANKRD36BP1, CCL20, AK056396, FAM35DP, SEPT3, CCL21, PDXP, LOC100132891, ABHD16B, LOC100288181, DMRT2, PPP1R36, FAM83A-AS1, TRAM1L1, GRIN2A, SOWAHA, LOC729970, LOC100127983, SLC35F3, NPPC, HIF1A-AS2, DISP2, SERPINA11, MTUS2, ACADL, PCDH20, KBTBD12, SLITRK5, AX746725, CXCL2, ISM2, AADACL2, TDRD5, AK095621, DES, DIRAS1, LOC100289511, RNF182, CYP2B7P1, CR936677, IRX6, CES1P2, AK054845, REN, PEG10, DKK2, RTP1, IL19, KCNJ16, CTAGE15, SYT2, MMRN1, AP4B1-AS1, DNAH17, AK096066, BC041347, BC034424, DKFZp686D16130, BC150585, AB209185, DKFZp547K2416, CCDC39, and/or AJ515158, or any combination thereof.

In some embodiments, a gene of the plurality of genes that are differentially expressed comprises IFNGR1, STAT4, CTLA4, IL12A, IRF1, STAT1, GZMA, LAG3, CD28, CCL5, CD8A, IL12RB2, PRF1, CXCL9, and/or CXCL10, or any subset thereof comprising at least 10 genes, at least 11 genes, at least 12 genes, at least 13 genes, or at least 14 genes.

In some embodiments, a gene of the plurality of genes that are differentially expressed comprises ANO1, TMEM71, CCL26, CTSC, HRH1, ALOX15, SIDT1, POSTN, SLC26A4, HDC, LRRC31, CPA3, DPYD, TNFAIP6, NTRK1, HLF, CXCL1, CLC, B2M, COL8A2, CA2, CXCL6, DPP4, SERPINB4, DSG1, FCER1A, KRT14, and/or KRT16 or any subset thereof comprising at least 20 genes, at least 22 genes, at least 24 genes, at least 26 genes, or at least 27 genes.

In some embodiments, a gene of the plurality of genes that are differentially expressed comprises CDH26, LOXL4, CFI, CCL24, CH25H, KRTAP3-2, IVL, SPRR3, TPSAB1, MMP12, UBC, RASGRP1, MK167, TGFB1, VCAM1, FLG, TFRC, MMP9, GATA3, CTSS, EML5, IL13RA1, EDNRA, UPK1B, DHRS9, VIM, SPRR2D, COL3A1, COL1A1, TUBB, CEACAM7, SERPINE1, COL1A2, TNFSF13B, SPP1, FZD10, CCL5, KIAA1199, MMP2, TNC, SPRR2C, COL5A2, PPIA, CLTC, STATE, SH2D1B, HPRT1, CDH1, GUSB, FMO2, CLEC7A, CLDN7, and/or ALAS1. In some embodiments, any one, any two, any three, any four, any five, any six, any seven, any eight, any nine, or any ten of the genes of the plurality of genes that are differentially expressed set forth above can be omitted from the gene expression analysis.

In some embodiments, a gene of the plurality of genes that are differentially expressed comprises CTSC, ANO1, ORAI1, ATIP1345, C1orf74, GCNT2, TRPM6, AP2M1, NTRK1, TRAPPC3, PITRMI1, MFHAS1, CLNS1A, NDUFA4, PHLDB2, TNIP2, CA2, ID3, COX6C, and/or GLDC, or any subset thereof comprising at least 15 genes, at least 16 genes, at least 17 genes, at least 18 genes, or at least 19 genes. In some embodiments, a gene of the plurality of genes that are differentially expressed comprises CRISP3, C1orf177, SPINK8, NUCB2, ZNF416, AMACR, TP5313, AMFR, BC107108, MUC21, CRISP2, GYS2, SFTA2, PAQR8, A1F1L, SPRR3, NDUFA4L2, CSTB, KRT4, and/or AK097161, or any subset thereof comprising at least 15 genes, at least 16 genes, at least 17 genes, at least 18 genes, or at least 19 genes.

In some embodiments, a gene of the plurality of genes that are differentially expressed comprises GPR97, LRRC31, GCNT2, VSTM1, HK3, CCL26, CEBPE, ATP13A5, CCR3, TRPM6, CTSC, ANO1, SUSD2, SLC26A4-AS1, BCL2L15, MT-CO2, LITAF, SYNPO, AP2M1, and/or CLC, or any subset thereof comprising at least 15 genes, at least 16 genes, at least 17 genes, at least 18 genes, or at least 19 genes. In some embodiments, a gene of the plurality of genes that are differentially expressed comprises ZNF416, ENDOU, SEPT5-GP1BB, EPGN, CRISP3, C2orf16, HSPA2, C1orf177, UACA, SPINK8, EPB41L3, CCNYL1, NDUFA4L2, SFTA2, NCOA1, AMFR, TGM3, KRT13, DPCR1, and/or NUCB2, or any subset thereof comprising at least 15 genes, at least 16 genes, at least 17 genes, at least 18 genes, or at least 19 genes.

In some embodiments, a gene of the plurality of genes that are differentially expressed comprises BC043620, ARHGEF16, ATP13A5, MT-ATP6, RBM38, ARHGEF35, RHOT2, PPARGC1B, MRPL43, GIPC2, PP7080, GAD1, SCRN2, UQCC, FADD, LINC00116, ZNF48, NDUFB10, CLNS1A, and/or PHLDA3, or any subset thereof comprising at least 15 genes, at least 16 genes, at least 17 genes, at least 18 genes, or at least 19 genes. In some embodiments, a gene of the plurality of genes that are differentially expressed comprises EPB41L3, NUCB2, A2ML1, CRISP3, TMED3, ZNF426, KRT32, MAPK3, CRYAB, C1orf177, LAMB4, AIF1L, BC107108, TMEM57, TNFRSF11A, CSTB, DPCR1, VSIG10L, CRISP2, and/or ACPP, or any subset thereof comprising at least 15 genes, at least 16 genes, at least 17 genes, at least 18 genes, or at least 19 genes.

In some embodiments, a gene of the plurality of genes that are differentially expressed in a downward direction comprises ALOX15, CCL26, POSTN, NRXN1, and/or CCR3. In some embodiments, a gene of the plurality of genes that are differentially expressed in an upward direction comprises SPINK8 and/or DSG1.

In some embodiments, a gene of the plurality of genes that are differentially expressed comprises MUC5AC, CCL18, CCL13, FCER2, CCL11, IL33, PTGDS, TSLP, DPP4, IL25, IL4, IL13, IL5, GATA3, CCR4, CLC, CCR3, PTGDR2, TPSAB1, HDC, IL1RL1, GATA1, SIGLEC8, CMA1, POSTN, ALOX15, CCL24, HRH1, STATE, IL4R, MUC5B, ARG1, FCER1A, CCL17, and/or IL13RA, or any subset thereof comprising at least 38 genes, at least 30 genes, at least 32 genes, at least 33 genes, or at least 34 genes. In some embodiments, a gene of the plurality of genes that are differentially expressed comprises CDA, EMR4P, CPR97, SIGLEC10, CD500LB, IL5RA, SIGLEC8, RAB37, IL1RL1, TESC, TREML2, ADAM5, MMP25, DAPK2, TRPM6, TPSAB1, CPA3, TPSB2, AIM2, SFRP1, CADM1, SCIN, CFI, CCNT3, CDH25, NTN1, EDAR, CAPN14, CALNT4, SIDT1, PLA2G3, IFF02, HAS3, CDH3, ID3, MFHAS1, SLC10A1, SERPINB4, IGFBP3, SUSD2, TNFSF13, LHFPL2, CTSC, CCNT2, SH3RF2, LITAF, KCNJ2, MAP3K14, TMTC3, KITLG, SCK1, TMEM173, PDZK1P1, IGFL1, EML1, SPINK7, CNFN, ZNF365, BNIP3, ME1, PPP2R2C, ANKRD37, CCNYL1, HSPA2, SAMO5, RFK, ZNF101, ZNF555, ZNF662, KIF21A, KRTAP3-2, YOD1, ZNF02, PHACTR2, PALMD, CYP2C15, TFAP2B, BOC, PFN2, and/or FAM125A, or any subset thereof comprising at least 75 genes, at least 76 genes, at least 77 genes, at least 78 genes, or at least 79 genes. In some embodiments, a gene of the plurality of genes that are differentially expressed comprises CCL26, MUC5B, CLC, IL1RL1, HDC, IL13, FCER1G, GATA2, and/or KIT.

In some embodiments, a gene of the plurality of genes that are differentially expressed comprises FLG, DSG1, SPINK7, SPINK8, SPINK5, KLK7, HAS3, THBS1, MMP9, LOX, POSTN, TIMP1, HAS2, IL13, PDGFRA, and/or LTBP2, or any subset thereof comprising at least 10 genes, at least 11 genes, at least 12 genes, at least 13 genes, or at least 14 genes. In some embodiments, a gene of the plurality of genes that are differentially expressed comprises DSG1, SPINK5, SPINK7, and/or SPINK8.

In some embodiments, a gene of the plurality of genes that are differentially expressed comprises CCL26, CCR3, ANO1, and/or SPINK8.

In some embodiments, a gene of the plurality of genes that are differentially expressed comprises UPK1B, SH2D1B, CDH26, POSTN, and/or DSG1. In some embodiments, a gene of the plurality of genes that are differentially expressed comprises ALOX15. In some embodiments, a gene of the plurality of genes that are differentially expressed comprises CCL26 and/or CCR3. In some embodiments, a gene of the plurality of genes that are differentially expressed comprises POSTN. In some embodiments, a gene of the plurality of genes that are differentially expressed comprises MUC5B. In some embodiments, a gene of the plurality of genes that are differentially expressed comprises mKi67, several collagen genes, DSG1, and/or SPINK family members. In some embodiments, a gene of the plurality of genes that are differentially expressed comprises SPINK5, SPINK7, and/or SPINK8. In some embodiments, a gene of the plurality of genes that are differentially expressed comprises ANO1. In some embodiments, a gene of the plurality of genes that are differentially expressed comprises NRXN1 and/or NTRK1. In some embodiments, a gene of the plurality of genes that are differentially expressed comprises IL13, CCL17, CCL18, and/or CCL26. In some embodiments, a gene of the plurality of genes that are differentially expressed comprises K16 and/or MK167.

In some embodiments, the methods comprise screening of the dupilumab core gene signature against a whole transcriptome profile from a plurality of disease studies comprises performing a differential gene expression analysis on the whole transcriptome profile for each disease study in the plurality of disease studies. In some embodiments, the screening of the dupilumab core gene signature against a whole transcriptome profile from a plurality of disease studies comprises generating a normalized enrichment score (NES) for all diseases in the plurality of disease studies using the plurality of genes that are differentially expressed and are in the dupilumab treatment core gene signature.

In some embodiments, the NES comprises an NES for eosinophilic esophagitis (EoE-NES), a type 2 gene expression signature (type 2-NES) in EoE, and/or a DpxOme-EoE™ NES.

In some embodiments, the NES comprises an EoE-NES. In some embodiments, the NES comprises a type 2-NES. In some embodiments, the NES comprises a DpxOme-EoE™ NES.

In some embodiments, the methods comprise performing a differential gene expression analysis on the whole transcriptome profile for each disease study in the plurality of disease studies is performed for disease versus healthy controls.

In some embodiments, the plurality of disease studies comprises the Gene Expression Omnibus database or the ArrayStudio DiseaseLand database.

In some embodiments, the NES is generated using a gene set enrichment analysis tool that takes both positive and negative gene sets into consideration. In some embodiments, the NES is computed separately for positive and negative gene sets.

In some embodiments, the NES is generated by ordering the plurality of genes that are differentially expressed from the most positive (i.e., most up-regulated) to the most negative (i.e., most down-regulated) values to generate a ranked gene list (R+). In some embodiments, the NES is generated by identifying hits (i.e., the rank for genes in the core signature) independently for the positive (i.e., most up-regulated) gene set (S+) in R+, and the negative (i.e., most down-regulated) gene set (S−) in R−, wherein R− is the inversed ranking of R+ with inverted values. In some embodiments, the NES is generated by combining R+ and R− and reordering the values by keeping the hits for both S+ and S−. In some embodiments, the NES is generated by computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S—; rᵢ is the value for gene i, and p is the weight for r. In some embodiments, the NES is generated by determining an Enrichment Score (ES) as a maximum deviation from zero along the running score. In some embodiments, the ordering, identification, combining, computing, and determining disclosed in this paragraph is repeated with a random gene set for 1,000 times to compute the ES null distribution. In some embodiments, the random gene set is a randomly selected list of genes (same size as the original gene set) from the whole transcriptome. In some embodiments, the NES is generated as the ES divided by the arithmetic mean of ES null distribution.

In some embodiments, the methods comprise computing the statistical significance by comparing the observed ES to the null distribution or sample label (disease/healthy) permutations.

In some embodiments, the ordering the plurality of genes that are differentially expressed from the most positive (i.e., most up-regulated) to the most negative (i.e., most down-regulated) values to generate a ranked gene list (R+) comprises using log 2 fold-change or z score. In some embodiments, fold-change, statistic (e.g., Wald test, T test), signal to noise ratio (difference of means scaled by the standard deviation) can be used for 2 groups comparison. In some embodiments, gene expression value or z score can be used for single sample NES.

In some embodiments, R+ and R− are ranked by log 2 fold-change comparing the mean gene expression in disease samples to the mean gene expression in healthy samples.

In some embodiments, the methods comprise computing the NES for all disease studies using a ranked list for each disease study.

In some embodiments, a disease with significant NES is a disease suitable for treatment with dupilumab.

The present disclosure provides methods of identifying a subject having a disease or condition suitable for treatment with dupilumab. In some embodiments, the methods comprise generating a dupilumab treatment core gene signature. In some embodiments, the methods comprise screening the dupilumab core gene signature against a whole transcriptome profile from the subject. In some embodiments, the methods comprise determining whether the subject is suitable for dupilumab treatment.

In some embodiments, the methods comprise generating the dupilumab treatment core gene signature comprising determining differential gene expression of a dupilumab treatment group and a placebo treatment group for a plurality of treatment studies and identifying a plurality of genes that are differentially expressed.

In some embodiments, the methods comprise transforming the whole transcriptome profile from the subject into z-scores. In some embodiments, the methods comprise ranking the z-scores. In some embodiments, the methods comprise generating a normalized enrichment score (NES) for all ranked z-scores using the plurality of genes that are differentially expressed and are in the dupilumab treatment core gene signature.

In some embodiments, the methods comprise generating the NES using a gene set enrichment analysis tool that takes both positive and negative gene sets into consideration.

In some embodiments, the NES is generated by transforming each gene expression within the plurality of genes into a z-score and ordering the plurality of genes that are differentially expressed from the most positive (i.e., most up-regulated) to the most negative (i.e., most down-regulated) values to generate a value of R+. In some embodiments, the methods can use the gene expression value (without any transformation) for ranking. In some embodiments, the NES is generated by identifying hits independently for the positive (i.e., most up-regulated) gene set (S+) in R+, and the negative (i.e., most down-regulated) gene set (S−) in R−, wherein R− is the inversed ranking of R+ with inverted values. In some embodiments, the NES is generated by combining R+ and R− and reordering the values by keeping the hits for both S+ and S−. In some embodiments, the NES is generated by computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r. In some embodiments, the NES is generated by determining an Enrichment Score (ES) as a maximum deviation from zero along the running score. In some embodiments, the transforming, identification, combining, computing, and determining disclosed in this paragraph is repeated with a random gene set for 1,000 times to compute the ES null distribution. In some embodiments, the NES is generated as the NES as ES divided by the mean of ES null distribution.

In some embodiments, the NES is generated by computing the statistical significance by determining the 95^{th} percentile NES from healthy control samples.

In some embodiments, the NES is generated by computing the NES for all disease studies using a ranked list for each disease study. In some embodiments, the NES is generated by computing the NES for all disease studies in the plurality of disease studies using a ranked list for each disease study of the plurality of disease studies.

In some embodiments, when the NES of the subject is higher than the NES of a healthy control, the subject is suitable for dupilumab treatment.

The present disclosure provides methods of carrying out a clinical trial for dupilumab treatment of a disease, disorder, or condition, the method comprising using a dupilumab core gene signature as a clinical endpoint for the clinical trial.

In some embodiments, the dupilumab treatment core gene signature is generated by determining differential gene expression of a dupilumab treatment group and a placebo treatment group for a plurality of treatment studies and identifying a plurality of genes that are differentially expressed.

In some embodiments, the clinical trial comprises generating a normalized enrichment score (NES) for the dupilumab treatment core gene signature prior to initiation of treatment of a subject with dupilumab and at least one time point after initiation of treatment of a subject with dupilumab.

In some embodiments, when dupilumab treatment results in a decrease in the NES for the dupilumab treatment core gene signature to an acceptable value, the clinical endpoint has been achieved.

In some embodiments, the NES is generated by ordering the plurality of genes that are differentially expressed from the most positive (i.e., most up-regulated) to the most negative (i.e., most down-regulated) values to generate a value of R+. In some embodiments, the NES is generated by identifying hits independently for the positive (i.e., most up-regulated) gene set (S+) in R+, and the negative (i.e., most down-regulated) gene set (S−) in R−, wherein R− is the inversed ranking of R+ with inverted values. In some embodiments, the NES is generated by combining R+ and R− and reordering the values by keeping the hits for both S+ and S−. In some embodiments, the NES is generated by computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r. In some embodiments, the NES is generated by determining an Enrichment Score (ES) as a maximum deviation from zero along the running score. In some embodiments, the ordering, identification, combining, computing, and determining disclosed in this paragraph is repeated with a random gene set for 1,000 times to compute the ES null distribution. In some embodiments, the NES is generated as ES divided by the mean of ES null distribution.

In some embodiments, the NES is generated by computing the statistical significance by comparing the observed ES to the null distribution or sample label (disease/healthy) permutations.

In some embodiments, the ordering the plurality of genes that are differentially expressed from the most positive (i.e., most up-regulated) to the most negative (i.e., most down-regulated) values to generate a value of R+ comprises using log 2 fold-change to compare gene expression after dupilumab treatment to gene expression prior to initiation of treatment with dupilumab.

In some embodiments, a plurality of samples is obtained from the subject and the NES is generated for each sample.

The differential gene expression comprises quantification (i.e., a measurement based on potentially many RNAs) of RNA/transcript expression of at least one gene in a biological sample from a subject. As used herein, “gene” is meant to also capture non-coding genes/biotypes (e.g., long non-coding RNAs). In some embodiments, the differential gene expression comprises quantification of an RNA expression level(s) of at least one gene in a biological sample from a subject. In some embodiments, the differential gene expression comprises quantification of an RNA expression level(s) of at least 10 genes. In some embodiments, the differential gene expression comprises quantification of an RNA expression level(s) of at least 20 genes. In some embodiments, the differential gene expression comprises quantification of an RNA expression level(s) of a level of at least 30 genes. In some embodiments, the differential gene expression comprises quantification of an RNA expression level(s) of at least 40 genes. In some embodiments, the differential gene expression comprises quantification of an RNA expression level(s) of at least 50 genes. In some embodiments, the differential gene expression comprises quantification of an RNA expression level(s) of at least 60 genes. In some embodiments, the differential gene expression comprises quantification of an RNA expression level(s) of at least 70 genes. In some embodiments, the differential gene expression comprises quantification of an RNA expression level(s) of at least 80 genes. In some embodiments, the differential gene expression comprises quantification of an RNA expression level(s) of at least 90 genes. In some embodiments, the differential gene expression comprises quantification of an RNA expression level(s) of at least 100 genes. In some embodiments, the differential gene expression comprises quantification of an RNA expression level(s) of at least 125 genes. In some embodiments, the differential gene expression comprises quantification of an RNA expression level(s) of at least 150 genes. In some embodiments, the differential gene expression comprises quantification of an RNA expression level(s) of at least 175 genes. In some embodiments, the differential gene expression comprises quantification of an RNA expression level(s) of at least 200 genes. In some embodiments, the differential gene expression comprises quantification of an RNA expression level(s) of at least 300 genes. In some embodiments, the differential gene expression comprises quantification of an RNA expression level(s) of at least 400 genes. In some embodiments, the differential gene expression comprises quantification of an RNA expression level(s) of at least 500 genes. In some embodiments, the differential gene expression comprises quantification of an RNA expression level(s) of at least 600 genes. In some embodiments, the differential gene expression comprises quantification of an RNA expression level(s) of at least 700 genes. In some embodiments, the differential gene expression comprises quantification of an RNA expression level(s) of at least 800 genes. In some embodiments, the differential gene expression comprises quantification of an RNA expression level(s) of at least 900 genes. In some embodiments, the differential gene expression comprises quantification of an RNA expression level(s) of at least 1,000 genes. In some embodiments, the differential gene expression comprises quantification of an RNA expression level of at least 5,000 genes. In some embodiments, the differential gene expression comprises quantification of an RNA expression level of at least 10,000 genes. In some embodiments, the differential gene expression comprises quantification of an RNA expression level of at least 15,000 genes. In some embodiments, the differential gene expression comprises quantification of an RNA expression level of at least 20,000 genes. In some embodiments, the differential gene expression comprises quantification of an RNA expression level of at least 25,000 genes. In some embodiments, the differential gene expression comprises quantification of an RNA expression level of at least 30,000 genes.

In some embodiments, the at least one gene comprises a protein-coding gene, a non-coding gene, a long non-coding RNA, a mitochondrial rRNA, a mitochondrial tRNA, an rRNA, a ribozyme, a B-cell receptor subunit constant gene, and/or a T-cell receptor subunit constant gene, or any combination thereof. In some embodiments, the at least one gene comprises a protein-coding gene. In some embodiments, the at least one gene comprises a non-coding gene. In some embodiments, the at least one gene comprises a long non-coding RNA. In some embodiments, the at least one gene comprises a mitochondrial rRNA. In some embodiments, the at least one gene comprises a mitochondrial tRNA. In some embodiments, the at least one gene comprises an rRNA. In some embodiments, the at least one gene comprises a ribozyme. In some embodiments, the at least one gene comprises a B-cell receptor subunit constant gene. In some embodiments, the at least one gene comprises a T-cell receptor subunit constant gene.

In any of the embodiments described herein, the biological sample comprises a sample from an organ, a tissue, a cell, and/or a biological fluid from the subject. In some embodiments, the biological fluid comprises plasma, serum, lymph, semen, and/or a mucosal secretion. In some embodiments, the biological sample comprises blood, semen, saliva, urine, feces, hair, teeth, bone, tissue, or a buccal sample. In some embodiments, the biological sample is obtained from the subject by a biopsy.

In some embodiments, RNA expression can be determined in part by RNA sequencing. In some embodiments, RNA sequencing reads can be mapped to a genome. In some embodiments, the genome is the human genome. In some embodiments, the human genome is reference assembly GRCh38. In some embodiments, the RNA sequencing reads can be limited to those for at least one protein coding gene, at least one long non-coding RNA, at least one mitochondrial rRNA, at least one mitochondrial tRNA, at least one rRNA, at least one ribozyme, at least one B-cell receptor subunit constant gene, and/or at least one T-cell receptor subunit constant gene. In some embodiments, the RNA sequencing reads are not so limited. In some embodiments, the sequences can be mapped without strand specificity, with strand-specific reverse first-read mapping, or with strand-specific forward first-read mapping. In some embodiments, the sequences can be mapped using kallisto v0.45.0 with strand-specific reverse first-read mapping (Bray et al., Nat. Biotechnol., 2016, 34, 525). In some embodiments, transcript counts can be aggregated to gene counts. In some embodiments, the aggregation can be conducted using tximport (Soneson et al., F1000Research, 2015, 4, 1521).

In some embodiments, the determination of a subject's NES comprises determining the RNA expression level(s) of one or more genes in a biological sample from a subject, comparing this RNA expression with the RNA expression of a corresponding gene from a placebo treatment group, determining the relative difference in RNA expression, and integrating the changes in the individual RNA expression into an NES. In some embodiments, the determination of a subject's NES comprises determining the RNA expression level(s) of one or more genes in multiple biological samples from a subject, determining the relative difference in RNA expression across the multiple samples, and integrating the changes in the individual RNA expression into an NES. In some embodiments, the genes whose RNA expression level(s) are measured include protein-coding genes, long non-coding RNAs, mitochondrial rRNAs, mitochondrial tRNAs, rRNAs, ribozymes, B-cell receptor subunit constant genes, and/or a T-cell receptor subunit constant genes. In some embodiments, the relative difference in RNA expression of genes in the panel are compared to the relative difference in RNA expression of genes not in the panel.

The present disclosure provides methods of treating a subject having a disease or condition suitable for treatment with dupilumab, the methods comprising: a) identifying the subject as having a disease or condition suitable for treatment with dupilumab comprising: i) generating a dupilumab treatment core gene signature; ii) screening the dupilumab core gene signature against a whole transcriptome profile from the subject; and iii) determining whether the subject is suitable for dupilumab treatment; and b) administering dupilumab to the subject having a disease or condition suitable for treatment with dupilumab.

In some embodiments, the dupilumab treatment core gene signature comprises determining differential gene expression of a dupilumab treatment group and a placebo treatment group for a plurality of treatment studies, and identifying a plurality of genes that are differentially expressed.

In some embodiments, the plurality of treatment studies comprises eosinophilic esophagitis, atopic dermatitis, asthma, grass allergy, and chronic rhinosinusitis with nasal polyposis.

In some embodiments, the genes in the core gene signature identified from the differential gene expression are selected as having a fold-change ≥2, and/or a q<0.05 in ≥3 out of 5 treatment studies. In some embodiments, the fold-change comprises subtracting the changes in expression in the placebo treatment group from the dupilumab treatment group.

In some embodiments, the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab. In some embodiments, the differential gene expression for the asthma and grass allergy treatment studies are carried out by comparing the gene expression with allergen challenge to the gene expression without allergen challenge. In some embodiments, the differential gene expression is analyzed by a microarray or RNASeq. In some embodiments, the differential gene expression of the eosinophilic esophagitis, asthma, and grass allergy treatment studies is analyzed by RNASeq. In some embodiments, the differential gene expression of the atopic dermatitis and chronic rhinosinusitis with nasal polyposis treatment studies is analyzed by microarray.

In some embodiments, the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes.

In some embodiments, screening the dupilumab core gene signature against a whole transcriptome profile from the subject comprises: i) transforming the whole transcriptome profile from the subject into z-scores; ii) ranking the z-scores; and iii) generating a normalized enrichment score (NES) for all ranked z-scores using the plurality of genes that are differentially expressed and are in the dupilumab treatment core gene signature, thereby representing the dupilumab signature enrichment for the subject.

In some embodiments, the NES is generated using a gene set enrichment analysis tool that takes both positive and negative gene sets into consideration. In some embodiments, the NES is generated by: a) transforming each gene expression within the plurality of genes into a z-score, and ordering the plurality of genes that are differentially expressed from the most positive (i.e., most up-regulated) to the most negative (i.e., most down-regulated) values to generate a value of R+; b) identifying hits independently for the positive (i.e., most up-regulated) gene set (S+) in R+, and the negative (i.e., most down-regulated) gene set (S−) in R−, wherein R− is the inversed ranking of R+ with inverted values; c) combining R+ and R− and reordering the values by keeping the hits for both S+ and S—; d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r; e) determining an Enrichment Score (ES) as a maximum deviation from zero along the running score; f) repeat steps a)-e) with a random gene set for 1,000 times to compute the ES null distribution; and g) generating the NES as ES divided by the mean of ES null distribution.

In some embodiments, the method further comprises computing the statistical significance by determining the 95^{th} percentile NES from healthy control samples. In some embodiments, the method comprises computing the NES for all disease studies using a ranked list for each disease study. In some embodiments, when the NES of the subject is higher than the NES of a healthy control, the subject is suitable for dupilumab treatment.

The present disclosure also provides kits, and method of using the kits. The kits can be used, for example, to detect the presence or absence of genes encoding any one or more of ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, and/or mRNA molecules and/or cDNA molecules derived therefrom. In some embodiments, any one or more of the aforementioned genes (and/or mRNA molecules and/or cDNA molecules derived therefrom) can be detected in a subject prior to treatment with dupilumab. In some embodiments, any one or more of the aforementioned genes (and/or mRNA molecules and/or cDNA molecules derived therefrom) can be detected in a subject after treatment with dupilumab. In some embodiments, any one or more of the aforementioned genes (and/or mRNA molecules and/or cDNA molecules derived therefrom) can be detected in a subject prior to treatment with dupilumab and after treatment with dupilumab.

In some embodiments, the kit comprises a plurality of nucleic acid molecules, wherein the plurality of nucleic acid molecules comprise nucleotide sequences that are complementary to at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3. In some embodiments, the kit comprises a plurality of nucleic acid molecules, wherein the plurality of nucleic acid molecules comprise nucleotide sequences that are complementary to at least 15, at least 20, at least 25, at least 30, at least 35, at least 40, or at least 45 of the nucleic acid molecules encoding the aforementioned genes (and/or mRNA molecules and/or cDNA molecules derived therefrom).

In some embodiments, the nucleic acid molecules comprising nucleotide sequences that are complementary to at least ten of the nucleic acid molecules encoding the aforementioned genes (and/or mRNA molecules and/or cDNA molecules derived therefrom) are probes. In some embodiments, the nucleotide sequences of the probes comprise at least 75%, at least 80%, at least 85%, at least 90%, at least 95%, or 100% sequence complementarity to the target gene. In some embodiments, the probe is 8 to 100, 10 to 75, 12 to 50, 15 to 25, or 18 to 23 nucleotides in length.

In any of the embodiments described throughout the present disclosure, the probes can comprise a label. In some embodiments, the label is a fluorescent label, a radiolabel, or biotin.

In any of the embodiments described throughout the present disclosure, each of the plurality of nucleic acid molecules is linked to a solid support. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a multiwell plate, such as a standard 96-well type. In some embodiments, a multiwell glass slide can be employed that normally contains one array per well. In some embodiments, the solid support is a microarray. In some embodiments, the solid support is a chip. In some embodiments, the solid support is a bead.

The present disclosure also provides methods of detecting a plurality of nucleic acid molecules in a subject, wherein the plurality of nucleic acid molecules detected comprise at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3. The methods comprise: contacting a biological sample from the subject with a plurality of nucleic acid molecules comprising nucleotide sequences that are complementary to at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3; and detecting the presence or absence of the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3. Any of the kits described herein can be used in these methods. In addition, any of the probes, or combinations thereof, described herein can be used in these methods. Any of the biological samples described herein can be used in these methods.

In some embodiments, the amount of the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3 in the biological sample is determined.

In some embodiments, each of the plurality of nucleic acid molecules comprising nucleotide sequences that are complementary to the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3 are labeled, and the detection step comprises detecting the label.

In some embodiments, each of the plurality of nucleic acid molecules comprising nucleotide sequences that are complementary to the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3 is linked to a solid support. Any of the solid support described herein can be used in these methods.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for.

### EXAMPLES

### Example 1: Dupilumab Normalizes the Eosinophilic Esophagitis Disease Transcriptome

### Study Design

The disclosed data were collected as part of a multicenter, randomized, double-blind, parallel-group, placebo-controlled, phase 2 study of dupilumab in adults with active eosinophilic esophagitis (EoE). A complete description of the study design is provided in, for example, Hirano et al., Gastroenterology, 2020, 158, 111-22.

In the phase 2 study, subjects completed a 35-day screening period, followed by 1:1 randomization to receive subcutaneous injections of dupilumab 300 mg (loading dose of 600 mg on day 1) every week or matched placebo for 12 weeks, and a 16-week follow-up period. An independent data and safety monitoring committee conducted blinded monitoring of subject safety data. The primary endpoint for the study was Straumann Dysphagia Instrument (SDI) patient-reported outcome (PRO) score from baseline to week 10.

Phase 3 TREET consisted of three parts. In part A, patients were randomized (1:1) to subcutaneous dupilumab 300 mg qw or matched placebo for 24 weeks. All patients from part A who continued in part C (part A-C) then received dupilumab 300 mg qw for an additional 28 weeks. In part B, patients were randomized (1:1:1) to dupilumab 300 mg qw, dupilumab 300 mg q2w, or placebo qw. Patients from part B continued to part B-C (ongoing); the dupilumab group continued on the same dose regimen, and patients from the placebo group were re-randomized (1:1) to dupilumab 300 mg qw or q2w. Patients were followed off the study drug for 12 weeks. The coprimary endpoints were histological remission (defined by 6 eosinophils per high-power field [eos/hpf]) and change from baseline in Dysphagia Symptom Questionnaire [DSQ] score) at week 24 (Dellon et al., N. Engl. J. Med., 2022, 387, 2317-2330).

### Subjects

The phase 2 study enrolled adult subjects (18-65 years) with documented EoE who were nonresponsive to protein-pump inhibitors (PPIs) and had active esophageal inflammation at screening. Such patients were identified by determining a peak cell count of ≥15 eosinophils per high-power field (400× magnification of a 0.3 mm² field) as indicated by esophageal pinch biopsy specimens from at least 2 of 3 esophageal sites. These specimens were obtained by endoscopy performed no more than 2 weeks after at least 8 weeks' treatment with high-dose (or twice-daily dosed) PPIs. Subjects were also required to have a self-reported history of an average of 2 episodes of dysphagia per week in the 4 weeks before screening with an SDI PRO score 5 at screening and baseline as well as a documented history or presence of at least one type 2 comorbid atopic disease. Full inclusion and exclusion criteria have been published previously (see, Hirano et al., Gastroenterology, 2020, 158, 111-22).

Phase 3 TREET enrolled patients aged 12 years with EoE who were non-responsive to 8 weeks of high-dose PPIs, had a peak intraepithelial eosinophil count 15 eos/hpf in at least two of three biopsied esophageal regions, and a patient-reported history of an average of two or more episodes of dysphagia per week in the 4 weeks before screening (Dellon et al., N. Engl. J. Med., 2022, 387, 2317-2330). Eligible patients also had at least four episodes of dysphagia in the 2 weeks prior to baseline, documented by DSQ eDiary (at least two of which required liquids, coughing, gagging, vomiting, or medical attention to obtain relief), completed at least 11 of 14 days of the DSQ eDiary in the 2 weeks prior to baseline, and a baseline DSQ score 10 (Dellon et al., N. Engl. J. Med., 2022, 387, 2317-2330).

### RNA Sequencing and Analysis

Pinch biopsies for RNA analysis were collected and frozen in RNALater™ from the proximal, mid, and distal esophagus during the screening and week 12 endoscopy procedures. After RNA extraction, strand-specific RNA-seq libraries were prepared using a KAPA stranded mRNA-Seq Kit (KAPA Biosystems, Roche Sequencing and Life Sciences, MA, USA). After amplification, sequencing was performed on Illumina HiSeq® 2000 device (Illumina Inc., CA, USA) by multiplexed single-read run (80 bp, 40M reads). Reads were mapped to the human genome (National Center for Biotechnology Information GRCh37) using Array Studio software (OmicSoft, NC, USA). Differentially expressed genes were identified using the DESeq2 package.

Using a gene set enrichment analysis tool that takes both positive and negative gene sets into consideration (see, world wide web at “mathworks.com/matlabcentral/fileexchange/33599-gsea2”; Lim et al., Pac. Symp. Biocomput., 2009, 504-15), the top 50 most upregulated and top 50 most downregulated genes in EoE were used to generate a normalized enrichment score (NES). For single-sample NESs, gene expression profiles were first transformed into z scores, and single-sample NES was computed using the ranked z scores in each sample to represent the sample's overall disease signature score, denoted as EoE-NES.

### Gene Ontology Term Enrichment Analysis

Unbiased global transcriptome analysis was performed using Gene Set Enrichment Analysis (Wapenaar et al., Immunogenetics, 2007, 59, 349-57) with gene ontology (GO) biological process gene sets from the Molecular Signatures Database (MSigDB, c5.bp.v7.0; The Gene Ontology Consortium, Nucleic Acids Res., 2019, 47, D330-D338; Ashburner et al., Nat. Genet., 2000, 25, 25-29). Gene sets with a size >100 were prefiltered to ensure biological process specificity and the top GO terms were selected if the false discovery rate was <0.05 in both EoE versus healthy and post-dupilumab treatment versus baseline comparisons. Eosinophil-associated genes and mast-cell associated genes were derived from the literature (Esnault et al., PLoS One, 2013, 8, e67560; and Abonia et al., J. Allergy Clin. Immunol., 2010, 126, 140-149).

### Immunohistochemistry (IHC)

Esophageal biopsies were stained with the following IHC markers: proliferation marker (MIB-1), lymphocyte markers (CD4, CD8), mast cell markers (chymase, tryptase), Fc receptor for IgE marker (FceR1), and Langerhans cell marker (CD1a). Scanned IHC images were annotated individually by a board-certified veterinary pathologist and analyzed using HALO (version 2.2) image analysis software. Quantification of CD4 expression was done using an area quantification algorithm, and for the remaining markers (CD8, MIB-1, CD1a, chymase, tryptase, FceR1), a cytonuclear algorithm was used.

### Statistical Analysis

DESeq2 version 1.26.0 was used to perform differential expression analysis. For the phase 2 study, week 12 data were compared with baseline within the two arms (placebo and dupilumab 300 mg qw). For the phase 3 TREET study, week 24 and week 52 data were compared with baseline values within the three arms (dupilumab 300 mg qw, dupilumab 300 mg q2w, and placebo). The dupilumab 300 mg qw group included patients pooled from parts A and B for assessment at baseline and week 24 and all patients assigned to weekly dosing in part C for assessment at week 52. The dupilumab 300 mg q2w group included patients from part B for assessment at baseline and week 24, and all patients assigned to alternate-week dosing in part C for assessment at week 52. The placebo group included patients pooled from parts A and B for assessment at baseline and week 24. The placebo/dupilumab 300 mg qw group at 52 weeks included patients assigned to placebo in parts A and B who received dupilumab 300 mg qw only in part C.

Genes were considered to be significantly modulated by treatment (dupilumab or placebo) if thresholds of a 2-fold relative log change from baseline and a q-value 0.05 were reached, thereby reflecting adjustment for multiple testing. Pearson correlations were calculated between the published gene changes in EoE (disease versus healthy) and gene changes after dupilumab treatment (post- versus pre-treatment). For the IHC analysis, nominal P values were calculated using unpaired two-sided t-tests to compare the absolute change from baseline between the placebo and treatment groups.

### Transcriptome Analysis

Transcriptome results were available for 16 of the 24 placebo patients (67%) and 22 of the 23 dupilumab patients (96%) enrolled in the phase 2 clinical trial (NCT02379052). However, biopsy specimens were not available from 6 patients, who were excluded from the analysis (see, Hirano et al., Gastroenterology, 2020, 158, 111-22).

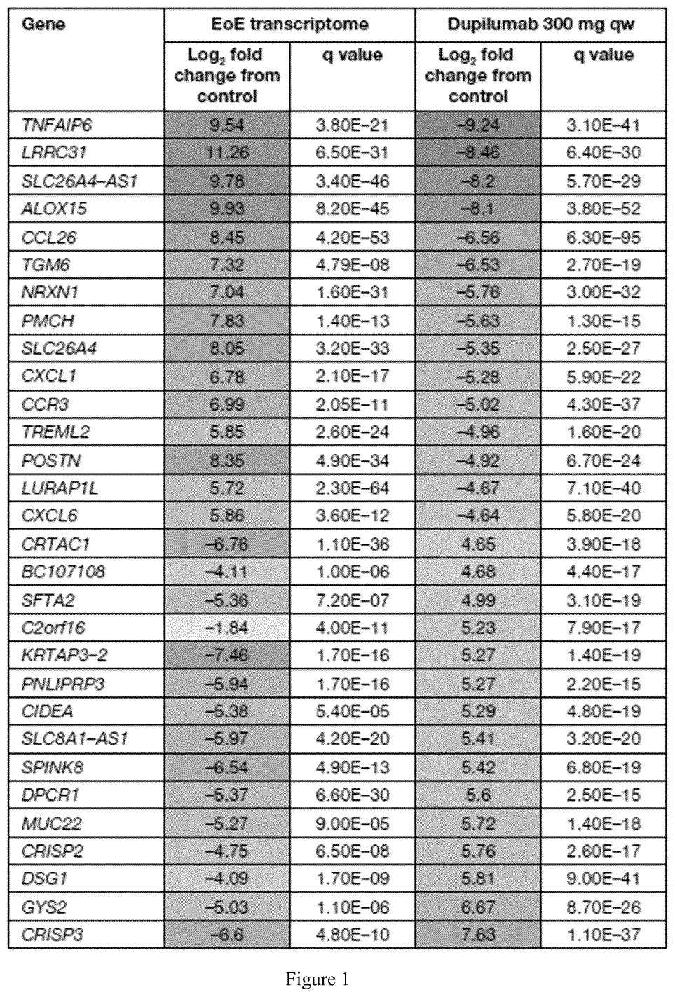

No genes were found to be differentially expressed (relative log change from baseline ≥2, q≤0.05) within the placebo arm at week 12 as compared with baseline expression levels. Dupilumab 300 mg weekly (qw) treatment modulated expression of 1,302 genes, the DpxOme-EoE™, at week 12 versus baseline, of which 513 were downregulated and 789 upregulated (heatmap data not shown). As the post-treatment results were highly similar across all 3 esophageal regions sampled (data not shown), mean values were presented across all samples (data not shown). The top 50 most upregulated and top 50 most downregulated genes in EoE were used to generate a normalized enrichment score (EoE-NES). Across the upregulated or downregulated genes, dupilumab treatment was associated with a significantly lower EoE-NES (Wilcoxon rank sum test, P<5.0×10⁻⁸). In contrast, no significant changes were associated with the placebo group. The 30 genes showing the highest changes in expression by dupilumab included those associated with type 2 inflammation, tissue remodeling/fibrosis, barrier function, and proliferation/differentiation (see, FIG. 1). Genes upregulated in the EoE transcriptome that were downregulated by dupilumab included ALOX15, CCL26, POSTN, NRXN1, and CCR3; genes downregulated in placebo subjects and upregulated by dupilumab included SPINK8 and DSG1.

Normalization of the EoE transcriptome

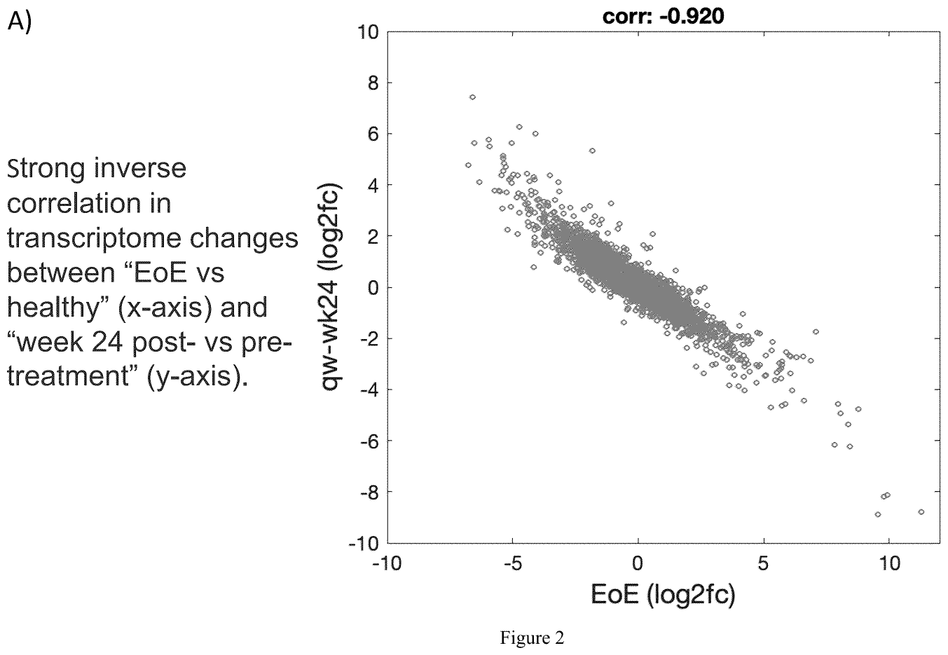

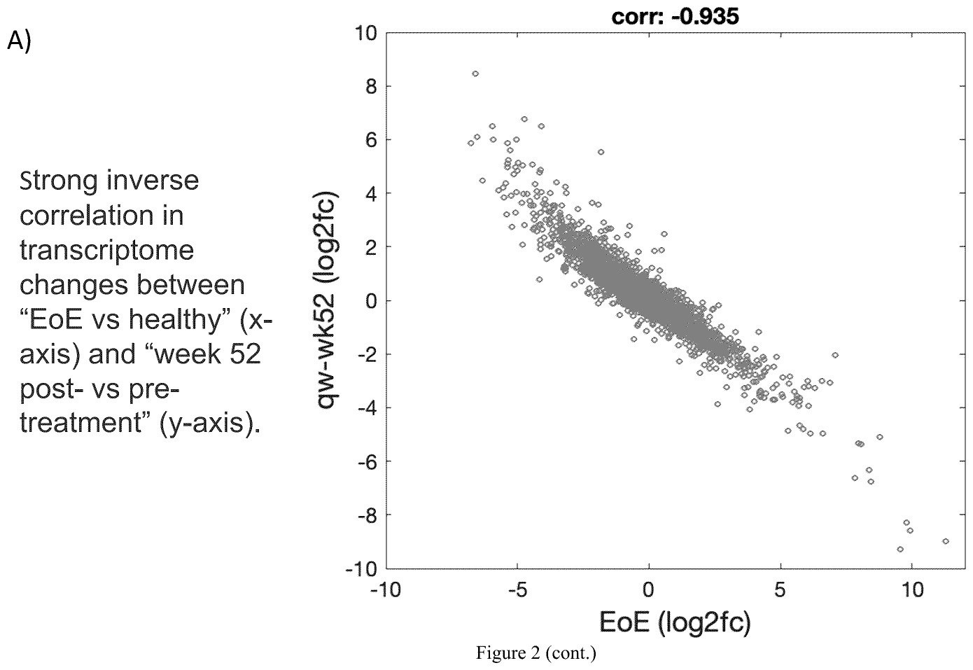

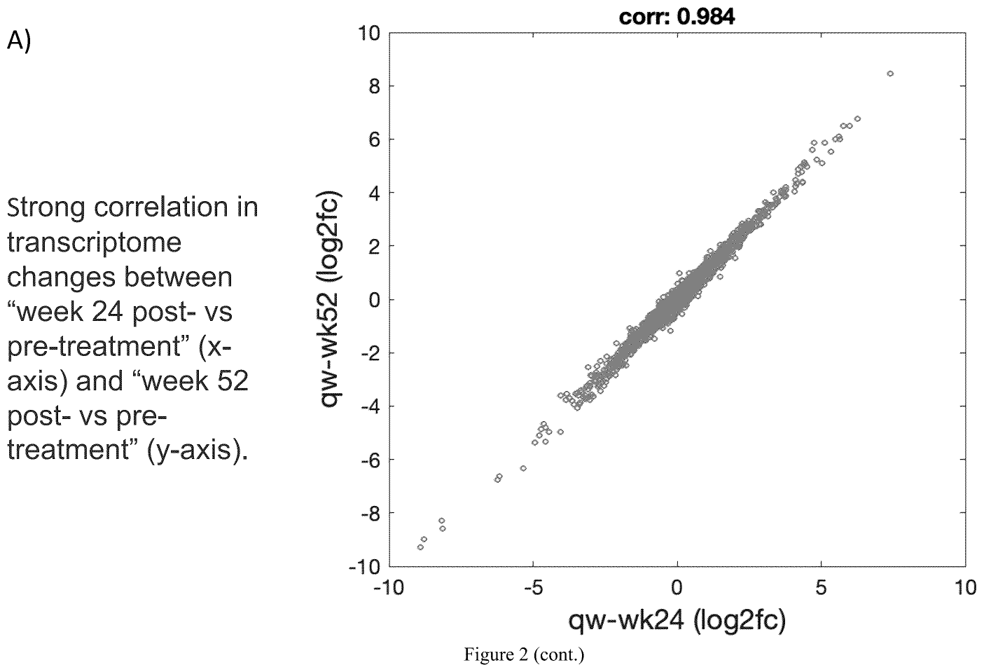

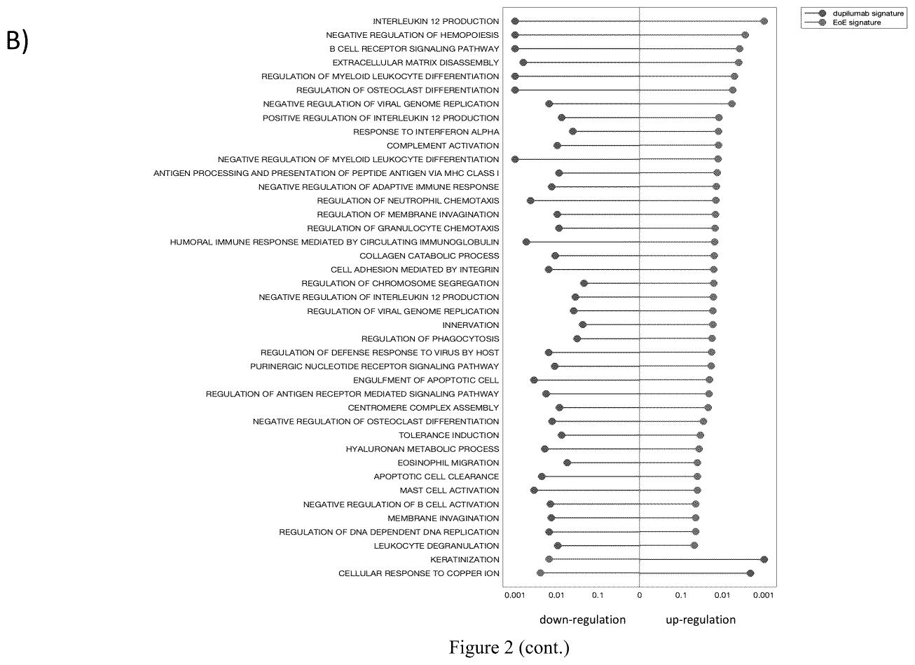

Treatment with dupilumab (week 12 versus baseline) normalized the transcriptome at week 12, as seen by comparison with the published EoE transcriptome and healthy transcriptome. As shown in FIG. 2 (Panel A), a strong negative correlation was observed between the published EoE transcriptome versus the healthy transcriptome and the DpxOme-EoE™ (week 12 versus baseline) (Pearson correlation coefficient: p=−0.872; P<1×10⁻⁶). There was a trend towards normalization for genes that did not meet the significance thresholds (heatmap data not shown). Dupilumab also significantly modulated a number of genes that were not included in the published EoE transcriptome (heatmap data not shown). Furthermore, many of the genes that did not overlap between the EoE and dupilumab signatures were not measurable in one or the other dataset. FIG. 2 (Panel B) shows that the main genes that are altered in EoE and modified by dupilumab treatment included the following GO groups: (i) immune function/inflammation (e.g. interleukin-12 production, B cell mediated immunity, response to type I interferon); (ii) eosinophil migration remodeling (e.g. extracellular matrix disassembly); (iii) mast cell activation; and (iv) epithelial differentiation (e.g. keratinization and cornification).

### Inflammation-Associated Genes

At week 12, dupilumab modulated type 2 inflammatory genes including IL4, IL13, IL13RA1, IL4R, IL5, IL33, TSLP, IL25, CCL11, CCL13, CCL17, CCL18, CCL24, CCL26, IL1RL1, FCER1A, FCER2, CCR3, CCR4, SIGLEC8, HDC, PTGDS, PTGDR2, CLC, ALOX15, MUC5B, MUC5AC, POSTN, DPP4, CMA1, TPSAB1, HRH1, GATA1, GATA3, ARG1, and STAT6 (heatmap data not shown). At week 12, dupilumab also modulated eosinophil-associated genes including ADAM8, CD300LB, DAPK2, EMR4P, GPR97, IL1RL1, IL5RA, MMP25, RAB37, SIGLEC10, SIGLEC8, TESC, TREML2, TRPM6, and CDA, and genes associated with mast cell activation including AIM2, CADM1, CAPN14, CDH26, CDH3, CFI, CPA3, CTSC, EDAR, GALNT4, GCNT2, GCNT3, HAS3, ID3, IFFO2, IGFBP3, KCNJ2, KITLG, LHFPL2, LITAF, MAP3K14, MFHAS1, NTN1, PDZK1IP1, PLA2G3, SCIN, SERPINB4, SFRP1, SGK1, SH3RF2, SIDT1, SLC16A1, SUSD2, TMEM173, TMTC3, TNFSF13, TPSAB1, TPSB2, ANKRD37, BNIP3, BOC, CCNYL1, CNFN, CYP2C18, EML1, FAM126A, HSPA2, IGFL1, KIF21A, KRTAP3-2, ME1, PALMD, PFN2, PHACTR2, PPP2R2C, RFK, SAMD5, SPINK7, TFAP2B, YOD1, ZNF101, ZNF365, ZNF555, ZNF662, and ZNF92 (data not shown). In particular, CCL26, MUC5B, CLC, IL1RL1, HDC, IL13, FCER1G, GATA2, and KIT were involved. Moreover, dupilumab treatment reduced eosinophil tissue infiltration at week 12 with similar effects on all 3 regions sampled (Hirano et al., Gastroenterology, 2020, 158, 111-22). Changes observed in eosinophil-associated gene expression were consistent with the decrease in density of eosinophils observed in esophageal biopsies after dupilumab treatment.

### Fibrosis, Remodeling, and Barrier Function Associated Genes

At week 12, dupilumab treatment also modulated genes associated with fibrosis, stroma remodeling, TGFβ and integrin signaling, such as collagen family genes and barrier-associated genes including FLG, DSG1, SPINK7, SPINK8, SPINK5, KLK7, HAS3, THBS1, MMP9, LOX, POSTN, TIMP1, HAS2, IL13, PDGFRA, and LTBP2 (heatmap data not shown). Of note, these genes included DSG1, SPINK5, SPINK7, and SPINK8.

### Transcriptomic Analyses Across Type 2 Inflammatory Diseases

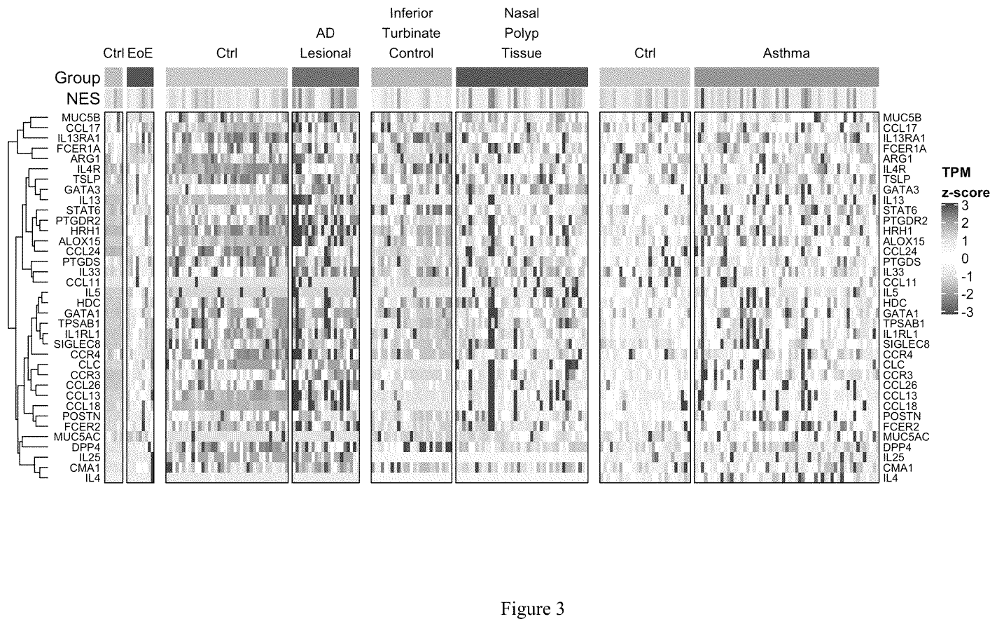

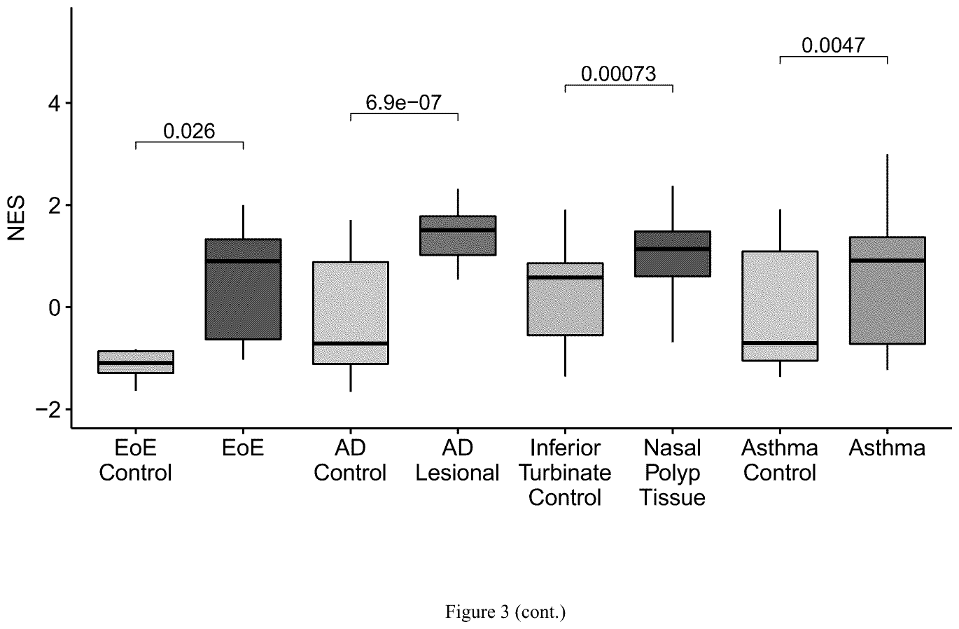

Because IL-4 and IL-13 are central mediators of type 2 inflammation, a type 2 gene expression signature (type 2-NES) in EoE was evaluated from the disclosed trial, other trials, and published studies of other atopic indications compared with relevant control tissues (heatmap data not shown). Although heterogeneity was evident in all of the indications, the type 2-NES was significantly higher in the disease groups relative to controls: EoE (P=0.026; Mishra et al., IL-5 promotes eosinophil trafficking to the esophagus, J. Immunol., 2002, 168, 2464-69), atopic dermatitis (GSE121212; P=6.9×10⁻⁷; Tsoi et al., J. Invest. Dermatol. 2019, 139, 1480-89), nasal polyps (GSE136825; P=0.00073; Peng et al., Eur. Respir. J., 2019, 54, 1900732) and asthma (GSE85567; P=0.0047; Nicodemus-Johnson et al., 2016, JCI Insight 1, e90151) (see, FIG. 3).

### Discussion

In the disclosed study, dupilumab significantly modulated the expression of 1,302 genes, reversing the disease transcriptional signature in EoE. Treatment with dupilumab led to a significantly lower EoE-NES (P<5.0×10⁻⁸), whereas no significant changes were observed in the placebo group. Dupilumab normalized the esophageal expression of genes dysregulated in EoE, including those involved in type 1 and 2 inflammation, fibrosis/remodeling, barrier function, mast cell activation, cell proliferation/differentiation, and eosinophilic inflammation, and those that are not normalized by corticosteroid treatment (UPK1B, SH2D1B, CDH26, POSTN, and DSG1; Nhu & Moawad, Curr. Treat. Options. Gastroenterol., 2019, 17, 48-62).

The most characteristic feature of EoE is infiltration by eosinophils into esophageal mucosa (Collins, Dig. Dis., 2014, 32, 68-73). Other pathologic changes associated with EoE include increased basal cell hyperplasia and dilated intercellular spaces, fibrosis of the lamina propria, and muscle hypertrophy (Guarino et al., World J. Gastrointest. Pharmacol. Ther., 2016, 7, 66-77). GO pathway analysis identified immune response and inflammation, eosinophil migration, and epithelial differentiation (cornification and keratinization) as the main pathways that are dysregulated in EoE and modulated by dupilumab, indicating the potential impact of dupilumab treatment on the histological changes and underlying inflammation associated with EoE. Previous studies on other biologic treatments targeting type 2 inflammatory responses in EoE have had mixed results, with some (e.g. anti-IL-13 and anti-IL-5 agents; Ko & Chehade, Clin. Rev. Allergy Immunol., 2018, 55, 205-16; Rothenberg al., J. Allergy Clin. Immunol., 135, 500-07; Hirano et al., Gastroenterology, 2019, 156, 592-603) resulting in histological improvements but no resolution of symptoms, and others (e.g. anti-IgE) having no impact (Clayton et al., Gastroenterology, 2014, 147, 602-09). No biologics other than dupilumab have yet demonstrated improvement in dysphagia symptoms in EoE trials, suggesting IL-4/IL-13 pathway effects of dupilumab are important in improving EoE disease severity. Furthermore, while IL-13 has been shown to induce EoE in murine models, mice deficient in IL-5, eotaxin-1, or STAT-6 demonstrated substantial protection against EoE development following IL-13 challenge (Mishra & Rothenberg, Gastroenterology, 2003, 125, 1419-27) indicating the interplay of a number of type 2 inflammatory components in disease pathogenesis.

Gene expression dysregulation in esophageal biopsies of EoE patients demonstrates abnormalities in not only type 2 inflammation, but also fibrosis/remodeling, impaired barrier function, and epithelial proliferation. Treatment with dupilumab reversed the type 2 inflammation observed in patients with active EoE. In the phase 2 study, dupilumab reduced the mean eosinophil count by 107% by week 12 and significantly reduced the peak intraepithelial eosinophil count (Hirano et al., Gastroenterology, 2020, 158, 111-22). The current analysis identified a number of EoE dysregulated genes associated with type 2 inflammatory responses that were modulated by dupilumab. One of the genes most highly modulated by dupilumab, ALOX15 (15-lipoxygenase), is a proinflammatory mediator upregulated in asthma (Clayton et al., Gastroenterology, 147, 602-09 (2014)). ALOX15 is regulated by both IL-4 and IL-13, which accounts for the substantial impact of dupilumab treatment on expression of this gene (Snodgrass & Brune, Front. Pharmacol. 2019, 10, 719). The most dysregulated gene in the published EoE transcriptome (Blanchard et al., J. Clin. Invest., 2006, 116, 536-47) eosinophil chemoattractant CCL26 (eotaxin-3), and its receptor, CCR3 (Pease & Williams, J. Allergy Clin. Immunol., 2019, 143, 552-53), were both downregulated by dupilumab in this study. The key role of eotaxins in eosinophil infiltration in EoE has been previously demonstrated in vivo in CCR3-deficient mice (Blanchard et al., J. Clin. Invest., 2006, 116, 536-47). POSTN (periostin), an extracellular matrix protein upregulated in EoE that likely plays a role in fibrosis as well as the type 2 inflammatory response, was significantly downregulated with dupilumab treatment, whereas it has been previously shown to remain dysregulated with corticosteroid treatment (Blanchard et al., J. Allergy Clin. Immunol., 2007, 120, 1292-1300; O'Dwyer & Moore, Cell. Mol. Life. Sci., 2017, 74, 4305-14). Studies in mice have indicated the role of POSTN in eosinophil recruitment to the esophagus in EoE (Blanchard et al., Mucosal Immunol., 2008, 1, 289-96). Other modulated genes associated with type 2 inflammation include MUC5B, which is downregulated in EoE but overexpressed in chronic rhinosinusitis with nasal polyps (Zhang et al., Allergy, 2019, 74, 131-40) and underexpressed in airways of asthma patients (Bonser & Erle, J. Clin. Med., 2017, 6, 112). Treatment with dupilunnab resulted in upregulation of MUC5B, leading to an expression pattern similar to healthy controls.

Similar to asthma, remodeling and fibrosis are critical clinical features of chronic EoE that contribute to symptoms and disease severity. Murine models have demonstrated the role of IL-5-mediated eosinophilia in fibrosis and tissue remodeling in EoE (Mishra et al., Gastroenterology, 2018, 134, 204-14). In the study disclosed herein, dupilunnab decreased expression of collagen genes and POSTN, demonstrating a direct effect on genes associated with fibrosis. Persistent eosinophilic tissue infiltration contributes to basal zone hyperplasia and development of lamina propria fibrosis. This results in physical esophageal changes, such as stiffness and changes in smooth muscle function. These changes in turn cause varying severity of clinical symptoms (Aceves, Dig. Dis., 2014, 32, 15-21). The molecular and histologic improvements in hallmarks of fibrosis and remodeling are consistent with the improvement in esophageal distensibility also observed in the disclosed trial (Hirano et al., Gastroenterology, 2020, 158, 111-22). Treatment with dupilunnab resulted in both symptomatic relief and visual improvements in endoscopic features in patients with active EoE (Hirano et al., Gastroenterology, 2020, 158, 111-22). Significant improvements in patients receiving dupilunnab compared with placebo were observed in EoE-EREFS, which a measure of endoscopically identified EoE esophageal mucosal inflammatory and remodeling features (P=0.0015). Significant improvements were also noted in histology severity and extent (EoE-HSS), which is a measure of eosinophil density, basal zone hyperplasia, eosinophil abscesses, eosinophil surface layering, surface epithelial alteration, dyskeratotic epithelial cells, and dilated intercellular spaces (P<0.0001; Hirano et al., Gastroenterology, 2020, 158, 111-22). The DpxOme™-EoE NES was highly correlated with the total EoE-HSS grade, demonstrating a biological association of the molecular signature with the clinical measure. The expression of CTSC was the most highly correlated single gene with total EoE-HSS, demonstrating the importance of proinflammatory proteolytic activity in the pathogenesis of the disease. A key signature of EoE is downregulation of the majority of genes involved in esophageal differentiation (Rochman et al., J. Allergy Clin. Immunol., 2007, 140, 738-49). In line with clinical findings, this analysis showed that dupilunnab normalized a number of genes involved in proliferation (hyperplasia), fibrosis, and epithelial barrier function. These included mKi67, several collagen genes, DSG1, and SPINK family members. The SPINK gene family is thought to be involved in protection against proteolytic degradation of the epithelium and mucosa (Wapenaar et al., Immunogenetics, 2007, 59, 349-57). While SPINK5 and SPINK7 have roles in regulation of barrier function, SPINK7 also regulates proinflammatory cytokine production (Azouz et al., Sci. Transl. Med., 2018, 10, eaap9736). SPINK8, the third-most highly expressed SPINK in the esophagus, is downregulated in EoE, but was upregulated by dupilumab in the current study, indicating that dupilumab also aids in restoration of epithelial barrier function in patients with active EoE.

In addition to improvements in epithelial barrier integrity and reduction in fibrosis, patients receiving dupilumab also showed significant improvements in dysphagia symptoms versus placebo (Hirano et al., Gastroenterology, 2020, 158, 111-22). ANO1, a calcium-activated chloride channel associated with gastric smooth muscle contraction and itch, is upregulated in esophageal biopsies in mouse models of EoE and patient samples, and was also modulated by dupilumab (Subramanian et al., Proc. Natl. Acad. Sci. U.S.A., 2005, 102, 15545-50; Vanoni et al., 2020, J. Allergy Clin. Immunol., 145, 239-54). Expression of ANO1, which has been correlated with eosinophil counts in EoE, and may also be associated with IL-13-induced basal cell proliferation (Vanoni et al., J. Allergy Clin. Immunol., 2020, 145, 239-54) and mucus hypersecretion (Lin et al., Exp. Cell Res., 2015, 334, 260-69) is induced by IL-4 in vitro, with elevated expression in allergic rhinitis (Kang et al., Am. J. Physiol. Lung Cell. Mol. Physiol., 2017 313, L466-L476). Other neurological-related genes identified in the published EoE transcriptome were also normalized by dupilumab, including NRXN1 and NTRK1, suggesting neuroinflammation regulated by IL-4 and/or IL-13 in EoE could contribute to dysphagia and other symptoms.

Dupilumab inhibits type 2 inflammatory cytokines IL-4 and IL-13 and blocks their proinflammatory signaling, which is implicated in numerous allergic diseases ranging from asthma to atopic dermatitis (Gandhi et al., Expert Rev. Clin. Immunol., 2007, 13, 425-37). In atopic dermatitis, treatment with dupilumab modified genes associated with type 2 inflammation (e.g. IL13, CCL17, CCL18, and CCL26), hyperplasia (K16 and MK167), epidermal differentiation and barrier and lipid metabolism (Hamilton et al., J. Allergy Clin. Immunol., 2014, 134, 1293-1300; Guttman-Yassky E. et al., J. Allergy Clin. Immunol. 2019, 143, 155-72), highlighting some of the similarities in the underlying pathogenesis of EoE and atopic dermatitis. Hyperplasia is also observed in other type 2 inflammatory conditions (e.g. epidermal thickening in AD), and treatment with dupilumab has been shown to reduce epidermal thickness and markers of proliferation (e.g. K16 and mKi67) in atopic dermatitis (Kang et al., Am. J. Physiol. Lung Cell. Mol. Physiol., 2017, 313, L466-L476). Additional analysis using the NES derived from the DpxOme™-EoE and type 2 inflammation signatures revealed upregulation of both signatures in the disease versus control samples across indications (Sherrill et al., Genes Immun., 2014, 15, 361-69; Tsoi et al., J. Invest. Dermatol. 2019, 139, 1480-89; Peng et al., Eur. Respir. J., 2019, 54, 1900732; Nicodemus-Johnson et al., 2016, JCI Insight 1, e90151), further demonstrating shared molecular pathogenesis despite the diverse tissue types and RNA profiling platforms used for these analyses.

Dupilumab, which inhibits IL-4 and IL-13, normalizes the transcriptome of esophageal pinch biopsies in patients with EoE, including genes related to inflammation, eosinophils, barrier function, and fibrosis, in line with study findings showing reduced histological disease characteristics and symptoms. The results suggest that dupilumab suppresses the type 2 inflammatory response in EoE, and aids in restoration of the esophageal mucosa. The impact of dupilumab on the combination of molecular features, symptoms and histopathological features in patients with active EoE suggests a key role for IL-4 and IL-13 in EoE pathogenesis.

### Example 2: Generating Dupilumab Treatment Core Gene Signature

The criteria for core genes selection were a fold-change ≥2 and q<0.05 in ≥3 out of 5 treatment studies: i) eosinophilic esophagitis (EoE); esophagus biopsy gene expression profiled by RNAseq; ii) atopic dermatitis (AD); skin biopsy gene expression profiled by microarray; iii) asthma; bronchial allergen challenge with House Dust Mite (HDM); sputum gene expression profiled by RNAseq; and iv) grass; nasal challenge with Timothy Grass; nasal brushing gene expression profiled by RNAseq; v) chronic rhinosinusitis with nasal polyposis (CRSwNP); nasal brushing gene expression profiled by microarray.

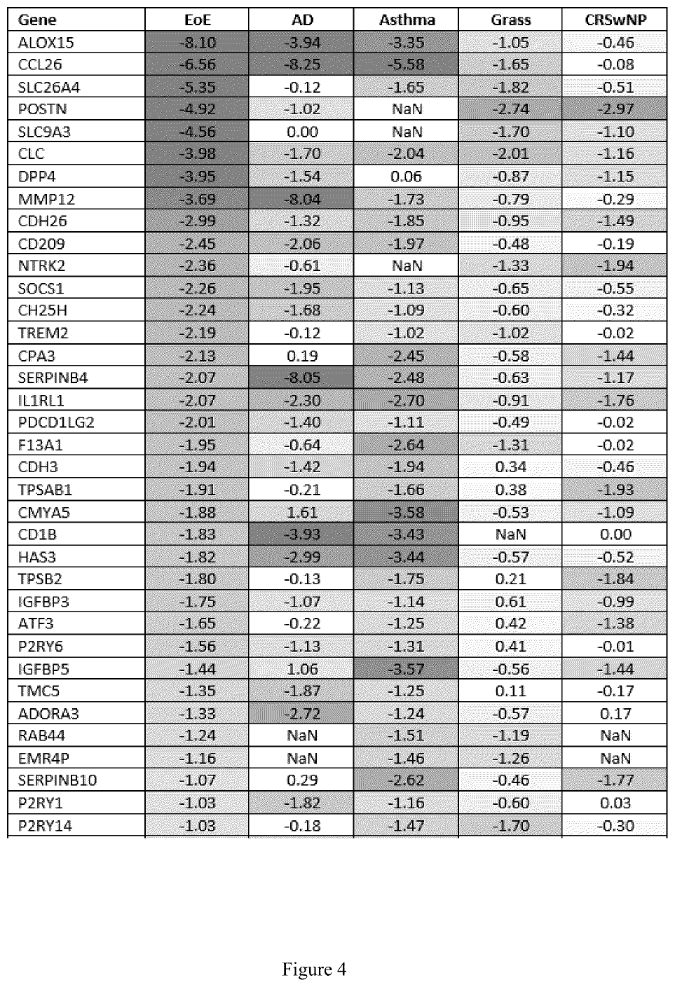

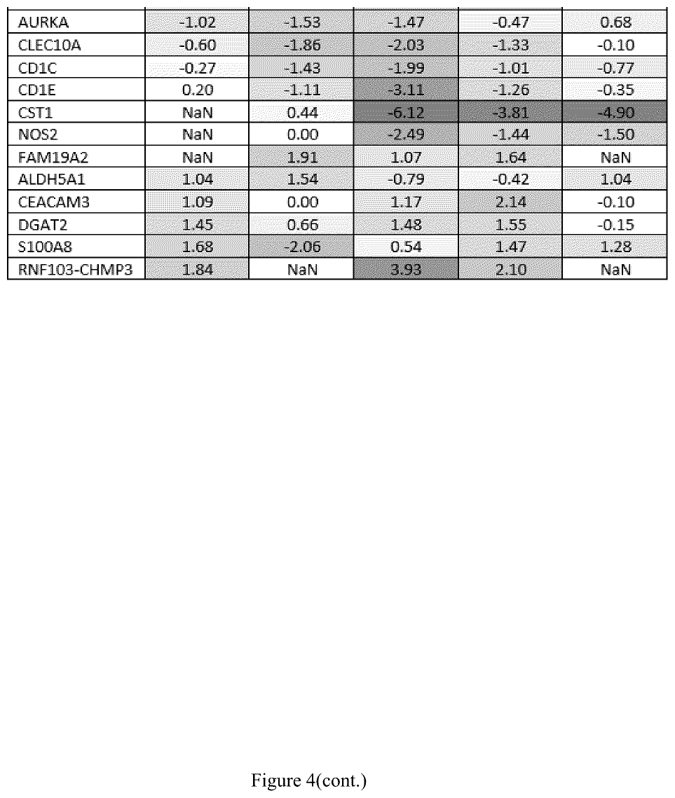

Differential gene expression was analyzed using limma package (Ritchie et al., Nuc. Acids Res., 2015, 43, e47) for microarray, and DESeq2 (Love et al., Genome Biol., 2014, 15, 550) for RNAseq. In the EoE/AD/CRSwNP studies, analyses were performed comparing baseline (before treatment) to after treatment. In the Asthma/Grass studies, treatment comparisons were performed with and without allergen challenge. Placebo-controlled treatment effect can be analyzed using differential foldchange analysis by subtracting the changes in placebo group from those in treatment group. Statistical significance can be determined by permutation testing, where the patient treatment assignment will be randomly permuted and a p value be computed by comparing the actual differential foldchange to the background distribution of the values from permutations. Results are shown in FIG. 4.

### Use Case 1: New Indication Screening

Whole transcriptome profiling technology have become increasingly popular and affordable. The amount of data generated and shared on public domain (e.g. Gene Expression Omnibus) has grown exponentially. Screening the dupilumab core signature on the large collection of disease studies could reveal previously unrecognized connections between the pathway perturbed by dupilumab and the disease mechanism. Diseases with differential gene expression (in opposite direction) of the dupilumab signature might represent potential new indications for the drug.

Using a gene set enrichment analysis tool that takes both positive and negative gene sets into consideration (Lim et al., Pac. Symp. Biocomput., 2009, 504-515), normalized enrichment score (NES) were generated for all disease studies from ArrayStudio DiseaseLand. Differential expression analyses were first performed in each study (disease vs healthy control). NES was then computed using genes on the whole transcriptome list ranked by fold-change to represent the enrichment of dupilumab signature in the study. The top hits from the screening include rhinitis, asthmatic rhinitis, allergic asthma, asthma, eosinophilic esophagitis, and atopic dermatitis. Some of these diseases were known indications of dupilumab where the disease signatures were reversed after treatments.

### Use Case 2: Patient Stratification

In disease where not all patients will benefit from dupilumab, the overall dupilumab NES will be diluted in the screening. In those diseases, the NES can be computer at an individual patient level. Instead of generating the NES on the whole transcriptome list ranked by disease-vs-healthy fold-change, gene expression profiles were first transformed into z-scores, and NES was computed using the ranked z-scores in each sample to represent the patient's dupilumab signature enrichment. Patients with higher NES could benefit more from dupilumab.

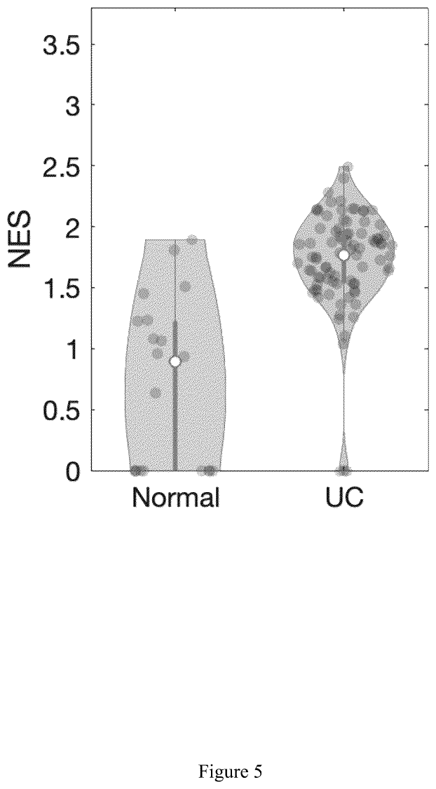

For instance, in the disease screening described earlier, ulcerative colitis (UC) NES is significantly enriched but not among the top 10. When NES was computed at the individual patient level using a UC study (GSE87466) with 87 patients and 21 healthy control, dupilumab NES were statistically significant in 33% of the patients. Results are shown in FIG. 5. Based on this analysis, in some embodiments, only this subset of patients with significant high NES score may be included in the population of patients for dupilumab treatment.

### Use Case 3: Molecular Pathology Endpoint

The differentially expressed genes in esophageal biopsies of EoE patients, as compared to healthy controls, is referred to as the EoE disease signature (Sherrill et al., Genes Immun., 2014, 15, 361-69). This disease signature was further refined into a smaller gene set to be used as EoE Diagnostic Panel (EDP) (Dellon et al., Clin. Transl. Gastroenterol., 2017, 8, e74). In the EoE phase 3 studies, the NES of the EDP genes was used as a secondary endpoint as an indicator of the disease activity. Dupilumab treatment, but not the placebo group, was shown to significantly decrease the NES. Comparable results were also observed using a gene signature representing type 2 inflammation that has been previously curated from the literature and preclinical experiments. Similar approaches can be generally applied to all indications using the dupilumab core gene signature to better capture the underlying molecular pathology activities and changes after dupilumab treatment.

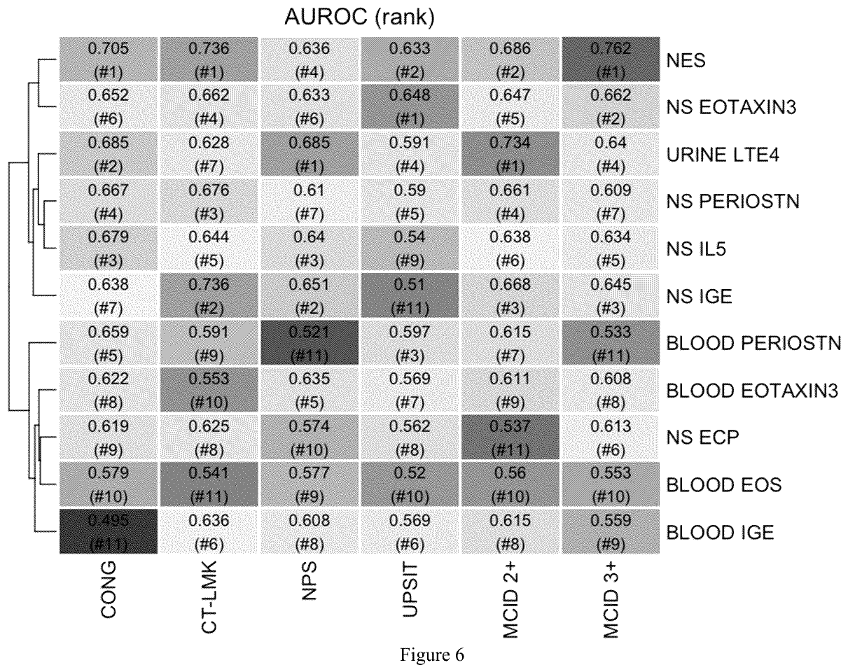

In the CRSwNP study, a set of 25 genes (identified from treatment and clinical response signature) was shown to be more predictive of response to each CRSwNP clinical endpoint (nasal congestion/obstruction (CONG), nasal poly score (NPS), Lund-Mackay computed tomography score (CT-LMK), and University of Pennsylvania Smell Identification Test (UPSIT)) than other available circulating biomarkers. A receiver operator characteristic analysis was used to assess the ability of the NES score representing the transcriptional signature and more standard biomarkers to discriminate the responders for each of the four major endpoints, as well as response across multiple endpoints. The predictive performance for each biomarker was summarized by calculating the area under the receiver operating characteristic curve (AUC). Results are depicted in FIG. 6. The dupilumab core gene signature highly overlapped with the 25 gene set and could be predictive of response to wider dupilumab indications.

### Example 3: Gene Signature Enrichment Score

Gene Sets Enrichment Analysis (GSEA) uses the Kolmogorov-Smirnov statistical test to assess whether a predefined gene set (in this case the dupilumab core signature) is statistically enriched in genes that are the two extremes of a list ranked by differential expression between two biological states (Subramanian et al., Proc. Natl. Acad. Sci. U.S.A. 2005, 102, 15545-50). The algorithm is very useful to detect differential expression of a set of genes collectively, even though the fold-change may be small for each individual gene. Since the dupilumab core signature include both up-regulated and down-regulated genes, the GSEA is extended to assess enrichment of two complementary gene sets against N ranked genes, as follows:
(a) Order the N genes from the most positive to the most negative values (e.g. log 2 fold-change, z score), denoted by R⁺.
(b) Identify hits independently for the positive gene set S⁺ in R⁺, and the negative gene set S⁻ in R⁻, in which R⁺ is the inversed ranking of R⁺ with the inverted values.
(c) Combine R⁺ and R⁻ and reorder the values by keeping the hits for both S⁺ and S⁻.
(d) Compute a running score by walking down the combined ranking. The score will increase by /rᵢ/^{p}/Σ_{i∈S}/r/^{p} if the i^{th} gene is a hit, or otherwise decrease by 1/(2N−S), where S is the combined total number of genes in S⁺ and S⁻. rᵢ is the value for gene i, and p is the weight for r.
(e) Enrichment Score (ES) is determined as maximum deviation from zero along the running score.
(f) Repeat steps (a-e) with random gene set for 1,000 times to compute the ES null distribution. Normalized Enrichment Score (NES) is determined as ES divided by the mean of ES null distribution.
(g) Statistical significance can be computed by comparing the observed ES to the null distribution.

In use case #1, R is ranked by log 2 fold-change comparing the mean gene expression in disease samples to the mean gene expression in healthy samples. There is a ranked list for each disease study, and NES can be computed for all studies. Diseases with significant NES will be deemed as potential indications for dupilumab. Statistical significance can be computed based on ES null distribution from random gene set (step f above), or sample label (disease/healthy) permutations.

In use case #2, gene expressions are transformed into z-score, gene by gene. In each sample, all genes are sorted by the z-score and used as the reference list, R, to compute NES. Patients with significant NES could be beneficial from dupilumab treatment. Statistical significance threshold can be determined as the 95^{th} percentile NES from the healthy control samples.

In use case #3, R can be ranked by log 2 fold-change comparing the gene expression after treatment to the baseline (before treatment) gene expression. NES is computed for each patient, denotes how the genes change with treatment. NES can be also computed for each sample, representing the overall expression level for the core signature in the sample.

### Example 4: Transcriptome Effects of Dupilumab are Durable

In parts A and B of the phase 3 TREET study (Dellon et al., N. Engl. J. Med., 2022, 387, 2317-2330), transcriptome results were available for 95 of 122 (78%) dupilumab 300 mg qw-treated patients, 60 of the 81 (74%) dupilumab 300 mg q2w-treated patients, and 84 of the 118 (71%) placebo-treated patients. In part C of the extended active treatment period, transcriptome results were available for 60 of 114 (53%) patients who continued on dupilumab 300 mg qw (dupilumab/dupilumab 300 mg qw) and 32 of 79 (41%) patients on dupilumab 300 mg q2w (dupilumab/dupilumab q2w), both up to week 52, and for 52 of 74 (70%) patients who switched from placebo to dupilumab 300 mg qw (placebo/dupilumab 300 mg qw), and 22 of 37 (59%) patients who switched to dupilumab 300 mg q2w (placebo/dupilumab 300 mg q2w) at the start of part C and received dupilumab for 24 weeks. For the phase 3 TREET study, results for the same dose regimens and time points are pooled across study parts.

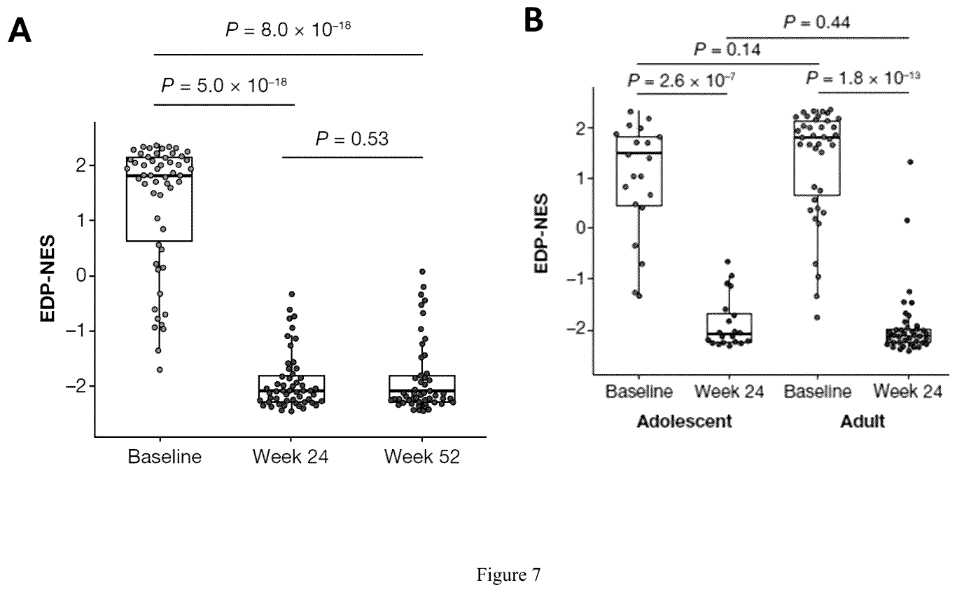

Using data from the phase 3 TREET study, changes in genes included in the EoE diagnostic panel (EDP; a further refined, smaller gene set compared with the EoE disease transcriptome) (Wen et al., Gastroenterology, 2013, 145, 1289-1299) were examined over time in patients treated with dupilumab 300 mg qw (see, FIG. 7, Panel A). EDP-NESs computed on the relative changes in pre- and post-treatments were included as secondary endpoints in the trial (Dellon et al., N. Engl. J. Med., 2022, 387, 2317-2330). Compared with baseline, a significant reduction in EDP was shown in dupilumab 300 mg qw-treated patients at week 24 (P=5.0×10⁻¹⁸), and was sustained in the dupilumab/dupilumab 300 mg qw group to week 52 (P=8.0×10⁻¹⁸), with no significant difference between the two time points (P=0.53). In general, the same patients whose EDP reduced at week 24 maintained suppression to week 52.

Transcriptome effects were seen in adolescents and adults. The EDP transcriptome was assessed in subgroups of adolescent and adult patients treated with dupilumab 300 mg qw in the phase 3 TREET study at baseline and week 24 to determine whether the impact of dupilumab treatment on the transcriptome was seen across age groups (see, FIG. 7, Panel B). A significant reduction in EDP-NES from baseline to week 24 was observed in both age groups (adolescents: P=2.6×10⁷; adults: P=1.8×10⁻¹⁸). EDP-NESs between adolescents and adults were not significantly different at baseline (P=0.14) and week 24 (P=0.44).

### Example 5: Dupilumab Suppressed MIB-1 (Ki67), Tryptase, and CD4⁺ T Cell Infiltration, and Moderately Increased CD1a⁺ Cells

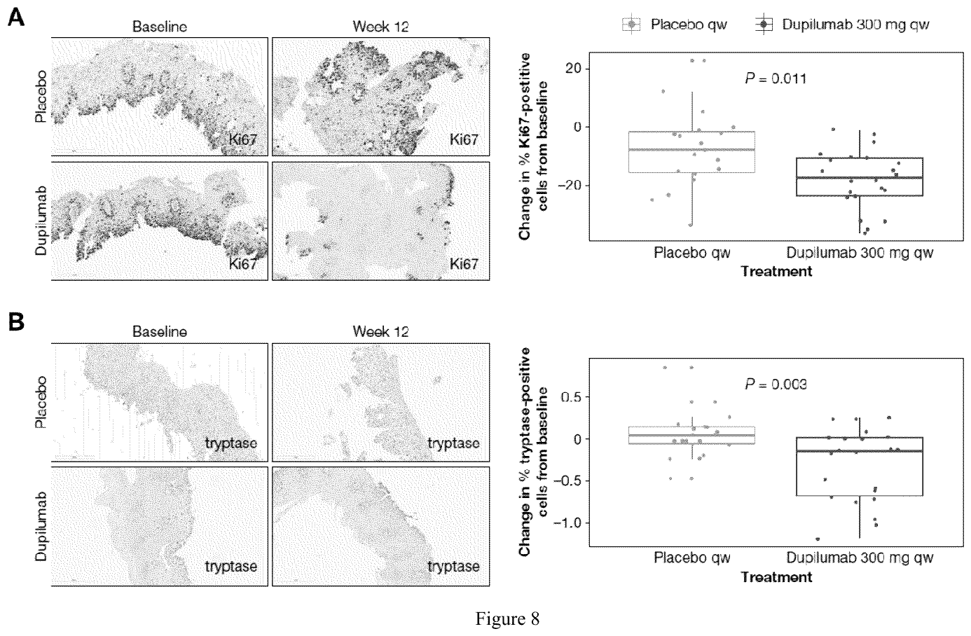

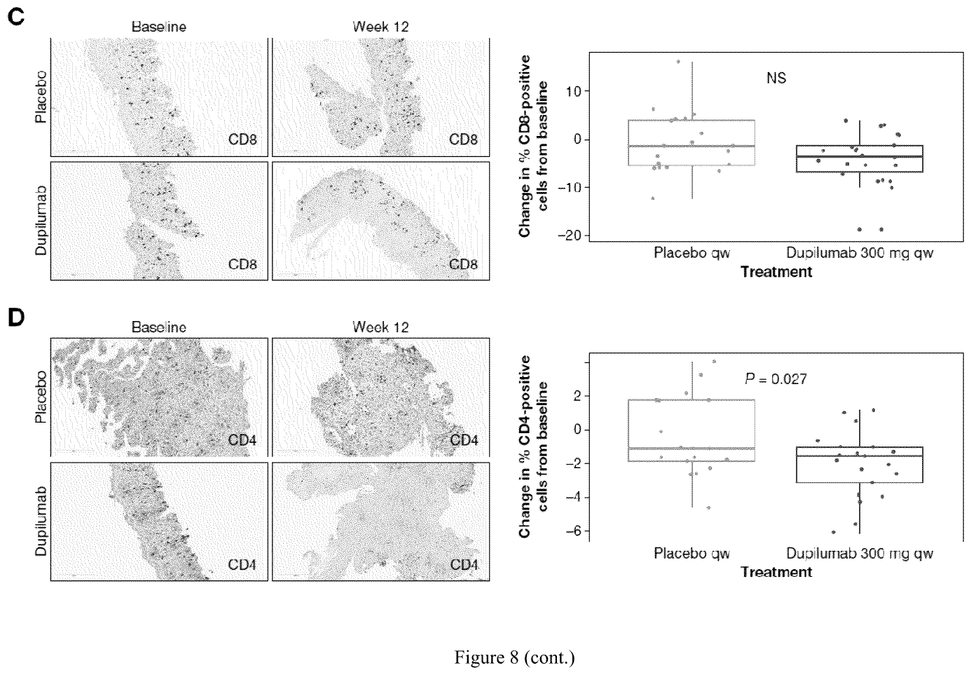

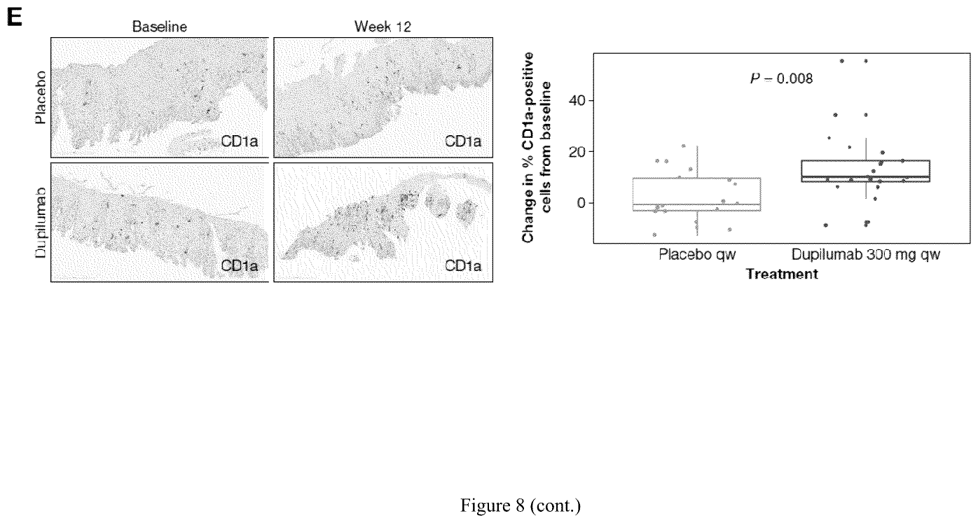

Prolonged eosinophil-predominant inflammation promotes epithelial hyperplasia in patients with EoE (Cheng et al., Am. J. Physiol. Gastrointest. Liver Physiol., 2012, 303, G1175-G1187), and dupilumab significantly decreased expression of MIB-1 (Ki67, a marker of cell proliferation) 12 weeks post-treatment compared with placebo treatment (P=0.011; see, FIG. 8, Panel A), consistent with observations of improved epithelial differentiation (by transcriptome analysis) and reduced basal zone hyperplasia in dupilumab-treated patients (Hirano et al., Gastroenterology, 2020, 158, 111-122). This is consistent with findings in patients with AD in whom dupilumab progressively reversed the characteristic epidermal hyperplasia (Guttman-Yassky et al., J. Allergy Clin. Immunol., 2019, 143, 155-172). Although the overall staining was low for tryptase, a marker of mast cell activation that is released upon mast cell degranulation, the number of cells expressing tryptase was reduced at week 12 in patients treated with dupilumab 300 mg qw compared with those receiving placebo (P=0.003; see, FIG. 8, Panel B). While no significant difference in CD8⁺ cytotoxic T cells was observed (see, FIG. 8, Panel C), dupilumab significantly reduced CD4⁺ cell (T helper) populations in patients compared with placebo (P=0.027; see, FIG. 8, Panel D). In contrast, CD1a⁺ cells (markers of antigen-presenting Langerhans cells) were increased with dupilumab treatment compared with placebo (P=0.008; see, FIG. 8, Panel E). No notable changes due to dupilumab treatment in the number of Fc∈RI- (Fc receptor for IgE) or chymase- (mast cell mediator; serine proteinases with chymotrypsin-like activity) positive cells were observed (data not shown).

### Example 6: Dpx3 Response Signature in EoE ph3

The Gene Set Enrichment Analysis (GSEA) Normalized Enrichment Score (NES) reflects the degree to which the activity level of a set of transcripts is overrepresented at the extremes (top or bottom) of the entire ranked list of transcripts. Gene expressions are transformed into z-score, gene by gene, and in each sample, all genes are sorted by the z-score and used as the reference list to compute single-sample NES. The NES calculated for the dupilumab response signature reflect the expression of a gene set in each esophageal biopsies from EoE patients to evaluate the molecular response. An NES that is closer to 0 indicates no response, and a negative score significantly deviated from 0 reflects a response.

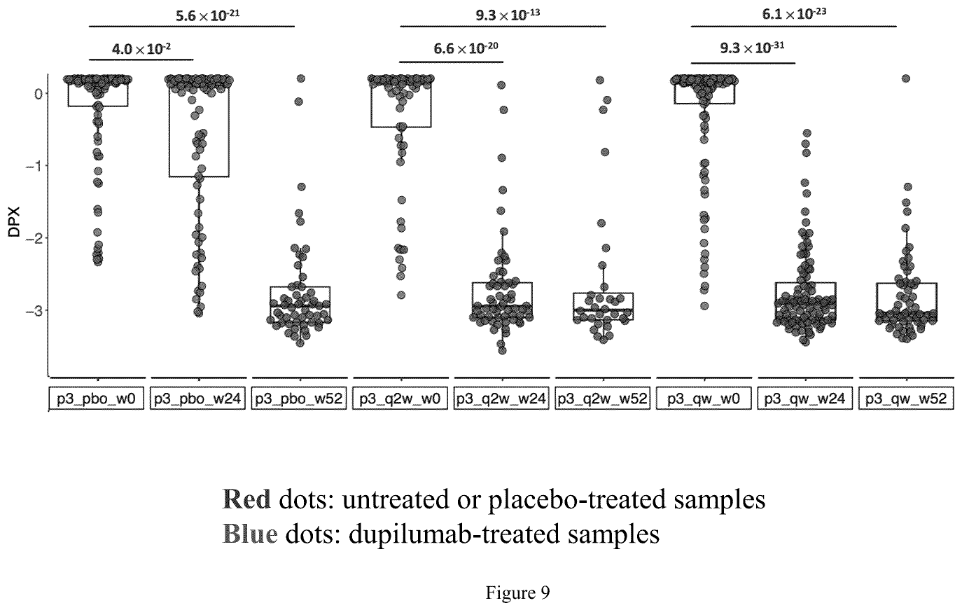

Both dupilumab 300 mg QW and Q2W show significant dupilumab molecular responses (strong negative NES) in EoE (see, FIG. 9). The average NES change from baseline to week 24 & 52 are: −0.32 (p=4.0×10⁻²) and −2.59 (5.6×10⁻²¹) in the placebo/dupilumab 300 mg group, −2.09 (6.6×10⁻²⁰) and −2.00 (9.3×10⁻¹³) in the dupilumab 300 mg Q2W/dupilumab 300 mg Q2W group, and −2.18 (9.3×10⁻³¹) and −2.22 (6.1×10⁻²³) in the dupilumab 300 mg QW/dupilumab 300 mg QW group. EoE: Eosinophilic esophagitis; NES: Normalized Enrichment Score; Q2W: Once every 2 weeks; QW: Once weekly.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

1-38. (canceled)

39. A method of carrying out a clinical trial for dupilumab treatment of a disease or condition, the method comprising using a dupilumab core gene signature as a clinical endpoint for the clinical trial.

40. The method of claim 39, wherein the dupilumab treatment core gene signature is generated by determining differential gene expression of a dupilumab treatment group and a placebo treatment group for a plurality of treatment studies, and identifying a plurality of genes that are differentially expressed.

41. The method of claim 40, wherein the plurality of treatment studies comprises eosinophilic esophagitis, atopic dermatitis, asthma, grass allergy, and chronic rhinosinusitis with nasal polyposis.

42-43. (canceled)

44. The method of claim 41, wherein the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab.

45-48. (canceled)

49. The method of claim 40, wherein the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes.

50. The method of claim 39, wherein the clinical trial comprises generating a normalized enrichment score (NES) for the dupilumab treatment core gene signature prior to initiation of treatment of a subject with dupilumab and at least one time point after initiation of treatment of a subject with dupilumab.

51. The method of claim 50, wherein when dupilumab treatment results in a decrease in the NES for the dupilumab treatment core gene signature to an acceptable value, the clinical endpoint has been achieved.

52-55. (canceled)

56. A method of treating a subject having a disease or condition suitable for treatment with dupilumab, the method comprising:
a) identifying the subject as having a disease or condition suitable for treatment with dupilumab comprising:
i) generating a dupilumab treatment core gene signature;
ii) screening the dupilumab core gene signature against a whole transcriptome profile from the subject; and
iii) determining whether the subject is suitable for dupilumab treatment; and
b) administering dupilumab to the subject having a disease or condition suitable for treatment with dupilumab.

57. The method of claim 56, wherein generating the dupilumab treatment core gene signature comprises determining differential gene expression of a dupilumab treatment group and a placebo treatment group for a plurality of treatment studies, and identifying a plurality of genes that are differentially expressed.

58. The method of claim 57, wherein the plurality of treatment studies comprises eosinophilic esophagitis, atopic dermatitis, asthma, grass allergy, and chronic rhinosinusitis with nasal polyposis.

59. The method of claim 57, wherein the genes in the core gene signature identified from the differential gene expression are selected as having a fold-change ≥2, and/or a q<0.05 in ≥3 out of 5 treatment studies.

60. The method of claim 59, wherein the fold-change comprises subtracting the changes in expression in the placebo treatment group from the dupilumab treatment group.

61. The method of claim 58, wherein the differential gene expression for the eosinophilic esophagitis, atopic dermatitis, and chronic rhinosinusitis with nasal polyposis treatment studies are carried out by comparing the baseline gene expression before treatment with dupilumab to the gene expression after treatment with dupilumab.

62. The method of claim 58, wherein the differential gene expression for the asthma and grass allergy treatment studies are carried out by comparing the gene expression with allergen challenge to the gene expression without allergen challenge.

63. The method of claim 57, wherein the differential gene expression is analyzed by a microarray or RNASeq.

64. The method of claim 63, wherein the differential gene expression of the eosinophilic esophagitis, asthma, and grass allergy treatment studies is analyzed by RNASeq.

65. The method of claim 63, wherein the differential gene expression of the atopic dermatitis and chronic rhinosinusitis with nasal polyposis treatment studies is analyzed by microarray.

66. The method of claim 57, wherein the plurality of genes that are differentially expressed comprises ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, or any subset thereof comprising at least 40 genes, at least 42 genes, at least 44 genes, at least 46 genes, or at least 47 genes.

67. The method of claim 56, wherein screening the dupilumab core gene signature against a whole transcriptome profile from the subject comprises:
i) transforming the whole transcriptome profile from the subject into z-scores;
ii) ranking the z-scores; and
iii) generating a normalized enrichment score (NES) for all ranked z-scores using the plurality of genes that are differentially expressed and are in the dupilumab treatment core gene signature, thereby representing the dupilumab signature enrichment for the subject.

68. The method of claim 67, wherein the NES is generated using a gene set enrichment analysis tool that takes both positive and negative gene sets into consideration.

69. The method of claim 68, wherein the NES is generated by:
a) transforming each gene expression within the plurality of genes into a z-score, and ordering the plurality of genes that are differentially expressed from the most positive (i.e., most up-regulated) to the most negative (i.e., most down-regulated) values to generate a value of R+;
b) identifying hits independently for the positive (i.e., most up-regulated) gene set (S+) in R+, and the negative (i.e., most down-regulated) gene set (S−) in R−, wherein R− is the inversed ranking of R+ with inverted values;
c) combining R+ and R− and reordering the values by keeping the hits for both S+ and S−;
d) computing a running score by walking down the combined ranking, wherein the running score increases by /rᵢ/^{p}/Σ_{i∈S}/r/^{p} if the i^{th} gene is a hit, or decreases by 1/(2N−S), where S is the combined total number of genes in S+ and S−; rᵢ is the value for gene i, and p is the weight for r;
e) determining an Enrichment Score (ES) as a maximum deviation from zero along the running score;
f) repeat steps a)-e) with a random gene set for 1,000 times to compute the ES null distribution; and
g) generating the NES as ES divided by the mean of ES null distribution.

70. The method of claim 69, further comprising computing the statistical significance by determining the 95^{th} percentile NES from healthy control samples.

71. The method of claim 69, the method comprising computing the NES for all disease studies using a ranked list for each disease study.

72. The method of claim 56, wherein when the NES of the subject is higher than the NES of a healthy control, the subject is suitable for dupilumab treatment.

73. A kit comprising a plurality of nucleic acid molecules, wherein the plurality of nucleic acid molecules comprise nucleotide sequences that are complementary to at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3.

74-75. (canceled)

76. A method of detecting a plurality of nucleic acid molecules in a subject after treatment with dupilumab, wherein the plurality of nucleic acid molecules detected comprise at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3, the method comprising:
contacting a biological sample from the subject with a plurality of nucleic acid molecules comprising nucleotide sequences that are complementary to at least ten of the nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3; and
detecting the presence or absence of the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3.

77. The method of claim 76, wherein the amount of the at least ten nucleic acid molecules encoding ALOX15, CCL26, SLC26A4, POSTN, SLC9A3, CLC, DPP4, MMP12, CDH26, CD209, NTRK2, SOCS1, CH25H, TREM2, CPA3, SERPINB4, IL1RL1, PDCD1LG2, F13A1, CDH3, TPSAB1, CMYA5, CD1B, HAS3, TPSB2, IGFBP3, ATF3, P2RY6, IGFBP5, TMC5, ADORA3, RAB44, EMR4P, SERPINB10, P2RY1, P2RY14, AURKA, CLEC10A, CD1C, CD1E, CST1, NOS2, FAM19A2, ALDH5A1, CEACAM3, DGAT2, S100A8, and RNF103-CHMP3 is determined.

78-80. (canceled)
